SEQUENCE LISTING (16 sequences)

SEQ ID NO: 1 - protein, 182 aa, Homo sapiens
MADSENQGPAEPSQAAAAAEAAAEEVMAEGGAQGGDCDSAAGDPDSAAGQMAEEPQTPAENAPKPKNDFIESLPNSVKCRVLALKKLQKRCDKIEAKFDKEFQALEKKYNDIYKPLLAKIQELTGEMEGCAWTLEGEEEEEEEYEDDEEEGEDEEEEEAAAEAAAGAKHDDAHAEMPDDAKK

SEQ ID NO: 2 - DNA, 2034 bp, Homo sapients
  CDS (95)...(643)
  misc_feature (1)...(17): vector sequence
gtcgacccacgcgtccggcaagatctctctggaccagctcgggtgcagggcctctgcgggagccctcctagacctctgcggcttctcctctaacatggccgactcggaaaaccaggggcctgcggagcctagccaggcggcggcagcggcggaggcagcggcagaggaggtaatggcggaaggcggtgcgcagggtggagactgtgacagcgcggctggtgaccctgacagcgcggctggtcagatggctgaggagccccagacccctgcagagaatgccccaaagccgaaaaatgactttatcgagagcctgcctaattcggtgaaatgccgagtcctggccctcaaaaagctgcagaagcgatgcgataagatagaagccaaatttgataaggaatttcaggctctggaaaaaaagtataatgacatctataagcccctactcgccaagatccaagagctcaccggcgagatggaggggtgtgcatggaccttggagggggaggaggaggaggaagaggagtacgaggatgacgaggaggagggggaagacgaggaggaggaggaggctgcggcagaggctgccgcgggggccaaacatgacgatgcccacgccgagatgcctgatgacgccaagaagtaaggggggcagagatggatgaagagaaagcccacgaagaaaaaagcctggttttgtttttcccagaatatcgatggacttaaaaaggctcaggtttttgaccaaaatacaatgtgaatttattctgacattcctaaaatagattaaattaaagcaattagatcctggccagctcgattcaaatttgactttcattttgaacataataaatatatcaaaaggtgttaaagaaaactgaattaaacccaaaattatgttttcatggtctcttctctgaggattgaggtttacaaagggtgttagcagatgcgaagtaaagaacgtcactttgaaacccattcatcacacagcatacgctacacatggaacacccaagccatgactgaacacgttctcagtgcttaattcttaaatttctttactcatgacatttcgcagtgcagagaaggcagaacccaagaaaaacgtcatctttgagactttgcttttgtaacgcagacatcagctttacacttcacaggagattgatggcattgaggaagattgcaatggagatcatgacactactgttaataaggccaggaaaactgccatttcaagttctgaaaaatgttttgagtatttgaatttagagaaacaacatggttccaagaaggagggtgtaaaacctgtaaaatactgtcaacatatgtattcattagttacaatctcatgtttgtgttttcttagtactgtctatttacaaacacgtaaaaaataccccaaatatgtttaagtattaaatcactttacctagcgttttagaaatattaatttacttgaagagatgtagaatgtagcaaattatgtaaagcatgtgtatccagcgttatgtactttgcgccttgtgacgtctttctgtcatgtagcttttagggtgtagctgtgaaaatcatcagaactcttcactgaagctaatgtttggaaaaaatatatacttgaagaaccaatccaagtgtgtgcccctacccccagctcagaagtagaaagggtttaagtttgcttgtattagctgtgccttcattattttgctatgtaaatgtgacatattaattataaaatggtgcataatcaaattttactgcttgaggacagatgcatacagtaaggatttttaggaagaatatatttaatgtaaagactcttagcttctgtgtgggttttgaattatgtgtgagccagtgatctataaagaaacataagcttaaagttgtttatcactgtggtgttaataaaacagtattttcaaaaaataaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagggcggccgc

SEQ ID NO: 3 - protein, 672 aa, Homo sapiens
MTPSQVAFEIRGTLLPGEVFAICGSCDALGNWNPQNAVALLPENDTGESMLWKATIVLSRGVSVQYRYFKGYFLEPKTIGGPCQVIVHKWETHLQPRSITPLESEIIIDDGQFGIHNGVETLDSGWLTCQTEIRLRLHYSEKPPVSITKKKLKKSRFRVKLTLEGLEEDDDDRVSPTVLHKMSNSLEISLISDNEFKCRHSQPECGYGLQPDRWTEYSIQTMEPDNLELIFDFFEEDLSEHVVQGDALPGHVGTACLLSSTIAESGKSAGILTLPIMSRNSRKTIGKVRVDYIIIKPLPGYSCDMKSSFSKYWKPRIPLDVGHRGAGNSTTTAQLAKVQENTIASLRNAASHGAAFVEFDVHLSKDFVPVVYHDLTCCLTMKKKFDADPVELFEIPVKELTFDQLQLLKLTHVTALKSKDRKESVVQEENSFSENQPFPSLKMVLESLPEDVGFNIEIKWICQQRDGMWDGNLSTYFDMNLFLDIILKTVLENSGKRRIVFSSFDADICTMVRQKQNKYPILFLTQGKSEIYPELMDLRSRTTPIAMSFAQFENLLGINVHTEDLLRNPSYIQEAKAKGLVIFCWGDDTNDPENRRKLKELGVNGLIYDRIYDWMPEQPNIFQVEQLERLKQELPELKSCLCPTVSRFVPSSLCGESDIHVDANGIDNVENA

SEQ ID NO: 4 - DNA, 3206 bp, Homo sapiens
  CDS (145)...(2163)
  misc_feature (1)...(17): Vector sequence
gtcgacccacgcgtccgggcgaggcacggacggcgggcgcccggtacctctgcccgcggtcctcgctctcgggcggggcggcggcgacgcggacctgcggactagcgaacccggagcacgacatcataaaataaatccatcagaatgacaccttctcaggttgcctttgaaataagaggaactcttttaccaggagaagtttttgcgatatgtggaagctgtgatgctttgggaaactggaatcctcaaaatgctgtggctcttcttccagagaatgacacaggtgaaagcatgctatggaaagcaaccattgtactcagtagaggagtatcagttcagtatcgctacttcaaagggtactttttagaaccaaagactatcggtggtccatgtcaagtgatagttcacaagtgggagactcatctacaaccacgatcaataacccctttagaaagcgaaattattattgacgatggacaatttggaatccacaatggtgttgaaactctggattctggatggctgacatgtcagactgaaataagattacgtttgcattattctgaaaaacctcctgtgtcaataaccaagaaaaaattaaaaaaatctagatttagggtgaagctgacactagaaggcctggaggaagatgacgatgatagggtatctcccactgtactccacaaaatgtccaatagcttggagatatccttaataagcgacaatgagttcaagtgcaggcattcacagccggagtgtggttatggcttgcagcctgatcgttggacagagtacagcatacagacgatggaaccagataacctggaactaatctttgattttttcgaagaagatctcagtgagcacgtagttcagggtgatgcccttcctggacatgtgggtacagcttgtctcttatcatccaccattgctgagagtggaaagagtgctggaattcttactcttcccatcatgagcagaaattcccggaaaacaataggcaaagtgagagttgactatataattattaagccattaccaggatacagttgtgacatgaaatcttcattttccaagtattggaagccaagaataccattggatgttggccatcgaggtgcaggaaactctacaacaactgcccagctggctaaagttcaagaaaatactattgcttctttaagaaatgctgctagtcatggtgcagcctttgtagaatttgacgtacacctttcaaaggactttgtgcccgtggtatatcatgatcttacctgttgtttgactatgaaaaagaaatttgatgctgatccagttgaattatttgaaattccagtaaaagaattaacatttgaccaactccagttgttaaagctcactcatgtgactgcactgaaatctaaggatcggaaagaatctgtggttcaggaggaaaattccttttcagaaaatcagccatttccttctcttaagatggttttagagtctttgccagaagatgtagggtttaacattgaaataaaatggatctgccagcaaagggatggaatgtgggatggtaacttatcaacatattttgacatgaatctgtttttggatataattttaaaaactgttttagaaaattctgggaagaggagaatagtgttttcttcatttgatgcagatatttgcacaatggttcggcaaaagcagaacaaatatccgatactatttttaactcaaggaaaatctgagatttatcctgaactcatggacctcagatctcggacaacccccattgcaatgagctttgcacagtttgaaaatctactggggataaatgtacatactgaagacttgctcagaaacccatcctatattcaagaggcaaaagctaagggactagtcatattctgctggggtgatgataccaatgatcctgaaaacagaaggaaattgaaggaacttggagttaatggtctaatttatgataggatatatgattggatgcctgaacaaccaaatatattccaagtggagcaattggaacgcctgaagcaggaattgccagagcttaagagctgtttgtgtcccactgttagccgctttgttccctcatctttgtgtggggagtctgatatccatgtggatgccaacggcattgataacgtggagaatgcttagtttttattgcacagaggtcattttgggggcgtgcaccgctgttctgggtattcatttttcatcactgagcattgttgatctatgccttttgggcttctcagttcaatgaagcaataatgaagtatttaactctttcactacagttcttgcaagtatgctatttaaattacttggccaggtataattgccagtcagtctctttatagtgagaaaatttattggttagtaatataaatattttaaactaaatatataaatctataatgttaaacatatgttcattaaaagcatagcactttgaaattaactatataaatagctcatatttacacttacagcttttcatttgatcaggtctgaaatctttagcacttaaggaaaatgactatgcataattatacctgaccatgaaaaaaataagtacctcaaatgcatgcatttgcactggtgattccaactgcacaaatctttgtgccatcttgtatataggtattttttacatgggttgacatgcacacaacaccattttcattcagtatgaaccttgaggctgctgccatttttccacttaaccaaaccagcctgaaggtgaacctcgaaacttgtttcataaatctttcaaaagttgttttacatcaatgttaaaatttcaaaatgctgcagggtaatttaatgtataaaatattagtaagaaaaagtatgtattgcatacttagtagaatagatcacaacatacaaattcaattcagtgcatgctttaggtgttaagcatgagattgtacatgtttactgttaggtccttgcatctgtggtgctaggtgagtatgagaagatgtcaaggactggacgtattttgttgcctaaaaaaaaaaggctgtttgtaggcgttttaaatatgcttattttgtgtgtctctcactacctattacacactgttgctttgtgggtttgttttgtatgtgcgtgtgttatacagtagttaaatttccatgcagaaaaataaatgtcctgaattctcaaaaaaaaaaaaaaagggcggccgc

SEQ ID NO: 5 - protein, 375 aa, Homo sapiens
MADHSFSDGVPSDSVEAAKNASNTEKLTDQVMQNPRVLAALQERLDNVPHTPSSYIETLPKAVKRRINALKQLQVRCAHIEAKFYEEVHDLERKYAALYQPLFDKRREFITGDVEPTDAESEWHSENEEEEKLAGDMKSKVVVTEKAAATAEEPDPKGIPEFWFTIFRNVDMLSELVQEYDEPILKHLQDIKVKFSDPGQPMSFVLEFHFEPNDYFTNSVLTKTYKMKSEPDKADPFSFEGPEIVDCDGCTIDWKKGKNVTVKTIKKKQKHKGRGTVRTITKQVPNESFFNFFNPLKASGDGESLDEDSEFTLASDFEIGHFFRERIVPRAVLYFTGEAIEDDDNFEEGEEGEEEELEGDEEGEDEDDAEINPKV

SEQ ID NO: 6 - protein, 375 aa, Mus musculus
MAENSLSDGGPADSVEAAKNASNTEKLTDQVMQNPQVLAALQERLDNVSHTPSSYIETLPKAVKRRINALKQLQVRCAHIEAKFYEEVHDLERKYAALYQPLFDKRREFITGDVEPTDAESAWHSENEEEDKLAGDMKNKVVIAEKEAATVEELNPKGIPEFWFTIFRNVDMLSELVQEYDEPILKHLQDIKVKFSDPGQPMSFVLEFHFEPNDYFTNPVLTKTYKMKSEPDKADPFSFEGPEIVDCDGCTIDWKKGKNVTVKTIKKKQKHKGRGTVRTITKQVPNESFFNFFSPLKASGDGESLDEDSEFTLASDFEIGHFFRERIVPRAVLYFTGEAIEDDDNFEEGEEGEEEELEGDEEGEDEDDADVNPKV

SEQ ID NO: 7 - protein, 460 aa, Homo sapiens
MAESENRKELSESSQEEAGNQIMVEGLGEHLERGEDAAAGLGDDGKCGEEAAAGLGEEGENGEDTAAGSGEDGKKGGDTDEDSEADRPKGLIGYVLDTDFVESLPVKVKYRVLALKKLQTRAANLESKFLREFHDIERKFAEMYQPLLEKRRQIINAIYEPTEEECEYKSDSEDCDDEEMCHEEMYGNEEGMVHEYVDEDDGYEDYYYDYAVEEEEEEEEEDDIEATGEENKEEEDPKGIPDFWLTVLKNVDTLTPLIKKYDEPILKLLTDIKVKLSDPGEPLSFTLEFHFKPNEYFKNELLTKTYVLKSKLAYYDPHPYRGTAIEYSTGCEIDWNEGKNVTLKTIKKKQKHRIWGTIRTVTEDFPKDSFFNFFSPHGITSNGRDGNDDFLLGHNLRTYIIPRSVLFFSGDALESQQEGVVREVNDAIYDKIIYDNWMAAIEEVKACCKNLEALVEDIDR

SEQ ID NO: 8 - protein, 460 aa, Mus musculus
MAESVDHKELSESNQEELGSQVMAEGPGESQDRSEGVSIEPGDGGQHGEETVAAGVGEEGKGEEAAAGSGEDAGKCGGTDEDSDSDRPKGLIGYLLDTDFVESLPVKVKCRVLALKKLQTRAAHLESKFLREFHDIERKFAEMYQPLLEKRRQIINAVYEPTEEECEYKSDCEDYFEEEMDEEEETNGNEDGMVHEYVDEDDGYEDCYYDYDDEEEEEEEDDSAGATGGEEVNEEDPKGIPDFWLTVLKNVEALTPMIKKYDEPILKLLTDIKVKLSDPGEPLSFTLEFHFKPNEYFKNELLTKTYVLKSKLACYDPHPYRGTAIEYATGCDIDWNEGKNVTLRTIKKKQRHRVWGTVRTVTEDFPKDSFFNFFSPHGISLNGGVENDDFLLGHNLRTYIIPRSVLFFSGDALESQQEGVVREVNDEIYDKIIYDDWMAAIEEVKACCKNLEALVEDIDR

SEQ ID NO: 9 - protein, 358 aa, Glycine max
MTNDNIAVTDLTSALNEENRADLVNALKSKIQSLAGAHSDVLETLSPNVRKRVESLREIQGKHDELEADFLKEREALEAKYQKLYQPLYTKRYEIVNGVTEVEGAANESTDESEENKEKGVPSFWLNAMENNDVLAEEISERDEGALKFLKDIKWSRIENPKGFKLDFFFDTNPYFSNTVLTKTYHMIDEDEPILEKAIGTEIEWYPGKCLTQKVLKKKPKKGSKNAKPITKTESCESFFNFFKPPEVPEDDADIDEDLAEELQNQMEQDYDIGSTLRDKIIPHAVSWFTGEAAQGDEFEDLEDDEDEEEDEDEDEDEEDDEDEDDEEEDDTKTKKKKSGKAQAGDGDGERPPECKQQ

SEQ ID NO: 10 - protein, 625 aa, Rattus norvegicus
MTPSQVTFEIRGTLLPGEVFAMCGNCDALGNWSPQNAVPLTESETGESVWKAVIVLSRGMSVKYRYFRGCFLEPKTIGGPCQVIVHKWETHLQPRSITPLENEIIIDDGQFGIHNGVETLDSGWLTCQTEIRLRLHFSEKPPVSITKKKFKKSRFRVKLTLEGLEEDDDDDDKASPTVLHKMSNSLEISLISDNEFKCRHSQPECGYGLQPDRWTEYSIQTMEPDNLELIFDFFEEDLSEHVVQGDVLPGHVGTACLLSSTIAESERSAGILTLPIMSRSSRKTIGKVRVDFIIIKPLPGYSCSMQSSFSKYWKPRIPLDVGHRGAGNSTTTAKLAKVQENTIASLRNAASHGAAFVEFDVHLSKDLVPVVYHDLTCCLTMKRKYEADPVELFEIPVKELTFDQLQLLKLSHVTALKTKDQKQCMAEEENSFSENQPFPSLKMVLESLPENVGFNIEIKWICQHRDGVWDGNLSTYFDMNAFLDIILKTVLENSGKRRIVFSSFDADICTMVRQKQNKYPILFLTQGKSDIYPELMDLRSRTTPIAMSFAQFENILGINAHTEDLLRNPSYVQEAKDKGLVIFCWGDDTNDPENRRKLKEFGVNGLIYDRYLFFVKNLHGIVQTV

SEQ ID NO: 11 - protein, 243 aa, Bacillus subtilis
LYIIAHRGASGYAPENTIAAFDLAVKMNADMIELDVQLTKDRQIVVIHDDRVDRTTNGSGFVKDFTLEELQKLDAGSWYGPAFQGERIPTLEAVLKRYHKKIGLLIELKGHPSQVGIEEEVGQLLGQFSFSINNIVQSFQFRSVQRFRELYPSIPTAVITRPNFGMLSRNQMKAFRSFANYVNIKHTRLNRLMIGSINKNGLNIFAWTVNNQKTAAKLQAMGVDGIVTDYPDFIIKDGKHENI

SEQ ID NO: 12 - protein, 358 aa, Escherichia coli K12
MKLTLKNLSMAIMMSTIVMGSSAMAADSNEKIVIAHRGASGYLPEHTLPAKAMAYAQGADYLEQDLVMTKDDNLVVLHDHYLDRVTDVADRFPDRARKDGRYYAIDFTLDEIKSLKFTEGFDIENGKKVQTYPGRFPMGKSDFRVHTFEEEIEFVQGLNHSTGKNIGIYPEIKAPWFHHQEGKDIAAKTLEVLKKYGYTGKDDKVYLQCFDADELKRIKNELEPKMGMELNLVQLIAYTDWNETQQKQPDGSWVNYNYDWMFKPGAMKQVAEYADGIGPDYHMLIEETSQPGNIKLTGMVQDAQQNKLVVHPYTVRSDKLPEYTPDVNQLYDALYNKAGVNGLFTDFPDKAVKFLNKE

SEQ ID NO: 13 - protein, 247 aa, Escherichia coli K12
MSNWPYPRIVAHRGGGKLAPENTLASIDVGAKYGHKMIEFDAKLSKDGEIFLLHDDNLERTSNGWGVAGELNWQDLLRVDAGSWYSKMFKGEPLPLLSQVAERCREHGMMANIEIKPTTGTGPLTGKMVALAARELWAGMTPPLLSSFEIDALEAAQQAAPELPRGLLLDEWRDDWRELTARLGCVSIHLNHKLLNKARVMQLKDAGLRILVYTVNKPQRAAELLRWGVDCICTDAIDVIGPNFTAQ

SEQ ID NO: 14 - protein, 256 aa, Mycobacterium tuberculosis
MEFLRHGGRIAMAHRGFTSFRLPMNSMGAFQEAAKLGFRYIETDVRATRDGVAVILHDRRLAPGVGLSGAVDRLDWRDVRKAQLGAGQSIPTLEDLLTALPDMRVNIDIKAASAIEPTVNVIERCNAHNRVLIGSFSERRRRRALRLLTKRVASSAGTGALLAWLTARPLGSRAYAWRMMRDIDCVQLPSRLGGVPVITPARVRGFHAAGRQVHAWTVDEPDVMHTLLDMDVDGIITDRADLLRDVLIARGEWDGA

SEQ ID NO: 15 - protein, 274 aa, Mycobacterium tuberculosis
MTWADEVLAGHPFVVAHRGASAARPEHTLAAYDLALKEGADGVECDVRLTRDGHLVCVHDRRLDRTSTGAGLVSTMTLAQLRELEYGAWHDSWRPDGSHGDTSLLTLDALVSLVLDWHRPVKIFVETKHPVRYGSLVENKLLALLHRFGIAAPASADRSRAVVMSFSAAAVWRIRRAAPLLPTVLLGKTPRYLTSSAATAVGATAVGPSLPALKEYPQLVDRSAAQGRAVYCWNVDEYEDIDFCREVGVAWIGTHHPGRTKAWLEDGRANGTTR

SEQ ID NO: 16 - protein, 241 aa, Mycoplasma pneumoniae
MLKRQLLLAHRGYSDIAPENTQLAFELAFQYRFDGVELDVHLTKDGELVIIHDETTTRTALVDKTIELETLASLKQDDHSAFFKFKTQPQPIMTLKEFFDQYLDKFQLINVEIKTDQKEYPGIEAKIDALAQQYGKKVIEKVVFSSFNFASLQRLYDINPNYQIAFLFWTKKQFQAVDALKIKQVCQYLHPWTNIYEKFPDMVLSLQLPLGLWTLNSEVKFHQFRQDRMVYAQIANKKFEV

(19) United States
(12) Patent Application Publication
(10) Pub. No.: US 2003/0165863 A1
(43) Pub. Date: Sep. 4, 2003

(54) NARC10 and NARC16, programmed cell death-associated molecules and uses thereof

(75) Inventor: Lillian Wei-Ming Chiang, Edison, NJ (US)

Correspondence Address: Intellectual Property Group, Millennium Pharmaceuticals Inc, 75 Sidney Street, Cambridge, MA, 02139, US

(73) Assignee: Millennium Pharmaceuticals, Inc.

(21) Appl. No.: 10/047,855

(22) Filed: Jan. 15, 2002

Related U.S. Application Data
(60) Provisional application No. 60/262,306, filed on Jan. 16, 2001.

(51) Int. Cl. C12Q 1/68; C07H 21/04; C12N 9/64; C12P 21/02; C12N 5/06

(52) U.S. Cl. 435/6; 435/69.1; 435/226; 435/320.1; 435/325; 536/23.2

(57) ABSTRACT

Novel programmed cell death-like polypeptides, proteins, and nucleic acid molecules are disclosed. In addition to isolated, full-length programmed cell death-related polypeptides, the invention further provides isolated programmed cell death-related fusion proteins, antigenic peptides, and anti-programmed cell death-related antibodies. The invention also provides programmed cell death-related nucleic acid molecules, recombinant expression vectors containing a nucleic acid molecule of the invention, host cells into which the expression vectors have been introduced, and nonhuman transgenic animals in which a programmed cell death-related gene has been introduced or disrupted. Diagnostic, screening, and therapeutic methods utilizing compositions of the invention are also provided.

### CROSS REFERENCE TO RELATED APPLICATIONS

This application claims the benefit of U.S. Provisional Application No. 60/262,306, filed Jan. 16, 2001.

### FIELD OF THE INVENTION

The invention relates to novel human programmed cell death-related sequences. Also provided are vectors, host cells, and recombinant methods for making and using the novel molecules.

### BACKGROUND OF THE INVENTION

In multicellular organisms, homeostasis is maintained by balancing the rate of cell proliferation against the rate of cell death. Cell proliferation is influenced by numerous growth factors and the expression of proto-oncogenes, which typically encourage progression through the cell cycle. In contrast, numerous events, including the expression of tumor suppressor genes, can lead to an arrest of cellular proliferation.

In differentiated cells, programmed cell death (apoptosis) occurs when an internal suicide program is activated. This program can be initiated by a variety of external signals as well as by signals that are generated within the cell in response to, for example, genetic damage. Dying cells are eliminated by phagocytes, without an inflammatory response.

Programmed cell death is a highly regulated process that involves transcription-dependent and -independent mechanisms (reviewed by Kaufman (1999) Genes Dev. 13:1211-1233; Dragunow and Preston (1995) Brain Res. Rev. 21:1-28; Raff et al. (1993) Science 286:2358-2361). A key molecular event in the process of programmed cell death is the activation of a signaling cascade mediated by the caspase family of serine proteases. Apoptotic signals include physiologic activators (growth factor deprivation, Fas activation, TGF-β, etc.), damage-related inducers (heat shock, viral infection, bacterial toxins, oncogenes, oxidants, free radicals, etc.) therapy-associated agents (chemotherapeutic drugs, ionizing radiation, etc.) and toxins (including ethanol and β-amyloid peptide). These signals result in the conversion of the precursors of the caspases into proteolytically active enzymes, resulting in the activation of late effectors of morphological and physiological aspects of programmed cell death, including DNA fragmentation and cytoplasmic condensation. In addition, regulation of programmed cell death may be integrated with regulation of energy, redox- and ion homeostasis in the mitochondria (reviewed by Kroemer (1998) Cell Death Differ. 5:547), and/or cell-cycle control in the nucleus and cytoplasm (reviewed by Choisy-Rossi et al. (1998) Cell Death Differ. 5:129-131; Dang (1999) Molec. Cell. Biol. 19:1-11; Kasten et al. Cell Death Differ. 5:132-140). 1998)).

Apoptotic cells undergo an orchestrated cascade of events including the activation of endogenous proteases, loss of mitochondrial function, and structural changes, such as disruption of the cytoskeleton, cell shrinkage, membrane blebbing, and nuclear condensation due to degradation of DNA. The various signals that trigger programmed cell death may bring about these events by converging on a common cell death pathway that is regulated by the expression of genes that are highly conserved.

Programmed cell death is a normal physiological activity required for proper growth and differentiation in all vertebrates. Defects in apoptotic programs result in disorders including, but not limited to, neurodegenerative disorders, cancer, viral infections, AIDS (acquired immunodeficiency syndrome), heart disease and autoimmune diseases (Thompson et al. (1995) Science 267:1456).

In neurons, programmed cell death is an essential component of development, and has been associated with many forms of neurodegeneration (reviewed by Hetts (1998) JAMA 279:300-307; Pettmann et al. (1998) Neuron 20:633-647; Jacobson et al. (1997) Cell 88:347-354). In vertebrate species, neuronal programmed cell death mechanisms have been associated with a variety of developmental roles, including the removal of neuronal precursors which fail to establish appropriate synaptic connections (Oppenheim et al (1991) Annual Rev. Neuroscience 14:453-501), the quantitative matching of pre- and post-synaptic population sizes (Herrup et al. (1987) J. Neurosci. 7:829-836), and sculpting of neuronal circuits, both during development and in the adult (Bottjer et al. (1992) J. Neurobiol. 23:1172-1191).

Inappropriate programmed cell death has been suggested to be involved in neuronal loss in various neurodegenerative diseases such as Alzheimer's disease (Loo et al. (1993) Proc. Natl. Acad. Sci. 90:7951-7955), Huntington's disease (Portera-Cailliau et al. (1995) J. Neurosc. 15:3775-3787), amyotrophic lateral sclerosis (Rabizadeh et al. (1995) Proc. Natl. Acad. Sci. 92:3024-3028), and spinal muscular atrophy (Roy et al. (1995) Cell 80:167-178).

In addition, improper expression of genes involved in programmed cell death has been implicated in carcinogenesis. Several families of oncogenes have been shown to play a role in programmed cell death. One example is the Bcl-2 family of proteins, which plays a pivotal role in the committing step of programmed cell death.

Accordingly, genes involved in programmed cell death are important targets for therapeutic intervention. It is important, therefore, to identify novel genes involved in programmed cell death or to discover whether known genes function in this process.

### SUMMARY OF THE INVENTION

Programmed cell-death related nucleotide sequences and polypeptides are provided. In particular, the present invention provides for isolated nucleic acid molecules comprising nucleotide sequences encoding the amino acid sequences shown in SEQ ID NO: 1 and SEQ ID NO: 3. Further provided are programmed cell death polypeptides having an amino acid sequence encoded by a nucleic acid molecule described herein.

The present invention also provides vectors and host cells for recombinant expression of the nucleic acid molecules described herein, as well as methods of making such vectors and host cells and using them for production of the polypeptides or peptides of the invention by recombinant techniques.

The disclosed invention relates to methods and compositions for the modulation, diagnosis, and treatment of disorders associated with dysregulated programmed cell death. By “dysregulated programmed cell death” is intended an alteration in the programmed cell death process that results in either an inappropriately low or high rate of programmed cell death.

Another aspect of this invention features isolated or recombinant programmed cell death-related polypeptides and polypeptides. Preferred programmed cell death polypeptides of the invention retain the activity of the reference programmed cell death polypeptides shown in SEQ ID NO: 1 or SEQ ID NO: 3.

Variant nucleic acid molecules and polypeptides substantially identical to the nucleotide and amino acid sequences set forth in the sequence listings are encompassed by the present invention. Additionally, fragments and substantially identical fragments of the nucleotide and amino acid sequences are provided.

Antibodies and antibody fragments that selectively bind the programmed cell death-related polypeptides and fragments are provided. Such antibodies are useful in detecting the programmed cell death-related polypeptides as well as in regulating cellular functions including programmed cell death, nucleosome assembly, phosphate homeostasis, and cell cycle.

In another aspect, the present invention provides a method for detecting the presence of programmed cell death-related activity or expression in a biological sample by contacting the biological sample with an agent capable of detecting an indicator of programmed cell death-related activity such that the presence of programmed cell death-related activity is detected in the biological sample.

In yet another aspect, the invention provides a method for modulating programmed cell death-related activity comprising contacting a cell with an agent or compound that modulates (inhibits or stimulates) programmed cell death-related activity or expression such that programmed cell death-related activity or expression in the cell is modulated. In one embodiment, the agent is an antibody that specifically binds to programmed cell death-related polypeptide. In another embodiment, the agent modulates expression of programmed cell death-related polypeptide by modulating transcription of a programmed cell death-related gene, splicing of a programmed cell death-related mRNA, or translation of a programmed cell death-related mRNA. In yet another embodiment, the agent is a nucleic acid molecule having a nucleotide sequence that is antisense to the coding strand of programmed cell death-related mRNA or programmed cell death-related gene.

In one embodiment, the methods of the present invention are used to treat a subject having a programmed cell death-related disorder characterized by programmed cell death-related polypeptide activity or nucleic acid expression by administering an agent or compound that is a modulator of programmed cell death to the subject. In one embodiment, the programmed cell death modulator is a programmed cell death-related polypeptide. In another embodiment, programmed cell death modulator is a programmed cell death-related nucleic acid molecule. In other embodiments, the programmed cell death modulator is a peptide, peptidomimetic, or other small molecule.

The present invention also provides a diagnostic assay for identifying the presence or absence of a genetic lesion or mutation characterized by at least one of the following: (1) aberrant modification or mutation of a gene encoding programmed cell death-related polypeptide; (2) misregulation of a gene encoding a programmed cell death-related polypeptide; and (3) aberrant post-translational modification of a programmed cell death-related polypeptide, wherein a wild-type form of the gene encodes a protein with a programmed cell death-related activity.

In another aspect, the invention provides a method for identifying a compound that binds to or modulates the activity of a programmed cell death-related polypeptide. In general, such methods entail measuring a biological activity of a programmed cell death-related polypeptide in the presence and absence of a test compound and identifying those compounds that alter the activity of the programmed cell death-related polypeptide.

The invention also features methods for identifying a compound or agent that modulates the expression of programmed cell death-related genes by measuring the expression of programmed cell death-related sequences in the presence and absence of the compound.

Other features and advantages of the invention will be apparent from the following detailed description and claims.

### BRIEF DESCRIPTION OF THE DRAWINGS

FIGS. 1A-D shows the amino acid sequence alignment for the protein (NARC10; SEQ ID NO: 1) encoded by human NARC10 (hNARC10C; SEQ ID NO: 2) with the human nucleosome assembly protein 1-like 4 (Q99733; SwissProt Accession Number Q99733; SEQ ID NO: 5), murine nucleosome assembly protein 1-like 4 (3319977; NCBI Accession Number AJ002198; SEQ ID NO: 6), human nucleosome assembly protein 2 (5931610; NCBI Accession Number BAA84706; SEQ ID NO: 7), murine nucleosome assembly protein 1-like 2 (P51860; NCBI Accession Number P51860; SEQ ID NO: 8), and soybean nucleosome assembly protein 1 (1161252; NCBI Accession Number AAA88792; SEQ ID NO: 9). The sequence alignment was generated using the Clustal method. The NARC10 protein shares approximately 26.4% identity with the human nucleosome assembly protein 1-like protein 4, approximately 25.8% identity with the murine nucleosome assembly protein 1-like protein 4, approximately 30.8% identity with the human nucleosome assembly protein 2, approximately 29.1% identity with the murine nucleosome assembly protein 1-like protein 2, and approximately 23.1% with the soybean nucleosome assembly protein 1, as calculated using the Clustal method with PAM250 residue weight table.

FIG. 2 shows a schematic diagram of the sequence similarity between human NARC16B and various related polypeptide sequences. The proteins shown are human NARC16B (SEQ ID NO: 3), rat NARC16 (SEQ ID NO: 10), D. melanogaster CG2818 protein (NCBI Accession No. AAF51071), C. elegans T05H10.7 protein (NCBI Accession No. Q10003), S. cerevisiae YPL110C protein (NCBI Accession No. NP_{—}015215), D. melanogaster CG9394 protein (NCBI Accession No. AAF46674), D. melanogaster CG11619 protein (NCBI Accession No. AAF49203), D. melanogaster CG3942 protein (NCBI Accession No. AAF54771), human MIR16 (NCBI Accession No. AF212862), and S. cerevisiae YPL206C protein (NCBI Accession No. S65225). Phylogenetically (as calculated using the Clustal method with the structural weight table), NARC16 is most closely related to the D. melanogaster CG2818 protein, and is also closely related to the C. elegans T05H10.7 and K10B3.6 proteins, the S. cerivisiae YPL110C protein, and the D. melanogaster CG11619 and CG9394 proteins. Conserved domains are indicated as follows: SBM=starch binding motif; AR=ankyrin repeat, GPDP=glycerol phosphodiester phosphodiesterase domain, C-term=conserved C-terminal domain.

FIGS. 3A-E shows the amino acid sequence alignment of amino acids 294-672 of NARC16 (SEQ ID NO: 3) with the Bacillus subtilus glycerophosphoryl phosphodiester phosphodiesterase (yhdW.seq; NCBI Accession Number E69827; SEQ ID NO: 11), Escherichia coli glycerophosphoryl phosphodiester phosphodiesterase (b2239.seq; NCBI Accession Number AAC75299; SEQ ID NO: 12), cytosolic Escherichia coli glycerophosphoryl phosphodiester phosphodiesterase (b3449; NCBI Accession Number AAC76474; SEQ ID NO: 13), Mycobacterium tuberculosis glycerophosphoryl phosphodiester phosphodiesterase glpQ2 (Rv0317c.seq; NCBI Accession Number CAB09602; SEQ ID NO: 14.), Mycobacterium tuberculosis glycerophosphoryl phosphodiester phosphodiesterase glpQ1 (Rv3842c.seq; NCBI Accession Number CAB06224; SEQ ID NO: 15), and Mycoplasma pneumoniae glycerophosphoryl phosphodiester phosphodiesterase (A05 orf241a.seq; NCBI Accession Number S73747; SEQ ID NO: 16). The sequence alignment was generated using the Clustal method. The NARC16 protein shares approximately 17.6% identity with B. subtilus glycerophosphoryl phosphodiester phosphodiesterase, 21.7% identity with E. coli glycerophosphoryl phosphodiester phosphodiesterase, 27.5% identity with cytosolic E. coli glycerophosphoryl phosphodiester phophodiesterase, 18.4% identity with M. tuberculosis glycerophosphoryl phosphodiester phosphodiesterase glpQ2, 23.8% identity with M. tuberculosis glycerophosphoryl phosphodiester phosphodiesterase glpQ1, and 22.3% sequence identity with M. pneumoniae glycerophosphoryl phosphodiester phosphodiesterase, as calculated using the Clustal method with PAM250 residue weight table.

FIGS. 4A-C provides the nucleotide and amino acid sequences for clone NARC10 (flhbNARC10C) and clone NARC16 (fthuNARC16B). The NARC10 nucleotide sequence is also set forth in SEQ ID NO: 2, and the NARC10 amino acid sequence is shown in SEQ ID NO: 1. The NARC16 nucleotide sequence is set forth in SEQ ID NO: 4, and the NARC 16 amino acid sequence is shown in SEQ ID NO: 3

FIG. 5 is a time course showing the percentage of Green Fluorescent Protein-expressing cells undergoing programmed cell death in cerebellar granular neurons transfected with an expression cassette encoding Green Fluorescent Protein (GFP), a GFP/caspase 4 positive control fusion protein (GFP.C4), a caspase 9/GFP positive control fusion protein (C9.GFP), or a GFP/NARC16 fusion protein (GFP.N16).

### DETAILED DESCRIPTION OF THE INVENTION

The present invention provides programmed cell death-related molecules. By “programmed cell death protein-like” is intended novel human sequence referred to as NARC10, or NARC16, and variants and fragments thereof. These full-length gene sequences or fragments thereof are referred to as “programmed cell death protein-like” sequences, indicating they play a role in programmed cell death. Isolated nucleic acid molecules comprising nucleotide sequences encoding the NARC10 polypeptide, whose amino acid sequence is given in SEQ ID NO: 1 and the NARC16 polypeptide, whose amino acid sequence is given in SEQ ID NO: 3 or variants or fragments thereof, are provided. A nucleotide sequence encoding the NARC10 polypeptide is set forth in SEQ ID NO: 2, and a nucleotide sequence encoding the NARC16 polypeptide is set forth in SEQ ID NO: 4.

Novel human programmed cell death-related gene sequences, referred to as NARC10 and NARC16, are provided. These gene sequences and variants and fragments thereof are encompassed by the term “programmed cell death protein-like” molecules or sequences as used herein. The programmed cell death-related sequences find use in modulating programmed cell death and cell proliferation. By “modulating” is intended the upregulating or downregulating of a response. That is, the compositions of the invention affect the targeted activity in either a positive or negative fashion. The activation of programmed cell death is manifested by changes including membrane blebbing, DNA fragmentation, cytoplasmic and nuclear degradation, chromatin aggregation, formation of apoptotic bodies, and cell death. Failure to appropriately activate programmed cell death can be manifested by changes including increased cell proliferation. Proteins and/or antibodies of the invention are also useful in modulating the apoptotic process.

The disclosed invention relates to methods and compositions for the modulation, diagnosis, and treatment of disorders associated with the inhibition of apoptosis, increased apoptosis, or with disruptions in the cell cycle.

Many disorders can be classified based on whether they are associated with abnormally high or abnormally low apoptosis. (Thompson (1995) Science 267:1456-1462). Apoptosis may be involved in acute trauma, myocardial infarction, stroke, and infectious diseases, such as viral hepatitis and acquired immunodeficiency syndrome.

Disorders associated with an inappropriately low rate of programmed cell death may prolong survival of abnormal cells (Thompson (1995) Science 267:1456-1462). These accumulated cells can give rise to cancers, including follicular lymphomas, carcinomas with p53 mutations, or hormone-dependent tumors, such as breast, prostate, or ovarian cancers. Autoimmune disorders, including systemic lupus erythematosus and immune-mediated glomerulonephritis, can arise if, for example, autoreactive lymphocytes are not removed following an immune response. Some viruses, including herepesviruses, poxviruses, and adenoviruses, have been shown to inhibit programmed cell death. Disabling this cellular defense mechanism allows the virus to propagate.

The molecules provided are also useful for the diagnosis and treatment of disorders associated with in creased levels of programmed cell death. These disorders are characterized by a marked loss of normal or protective cells and include (but are not limited to): virus-induced lymphocyte depletion (including AIDS); neurodegenerative diseases manifested by loss of specific sets of neurons (including Alzheimer's disease, Parkinson's disease, amyotrophic lateral sclerosis, retinitis pigmentosa, and cerebellar degeneration); myelodysplastic syndromes (including aplastic anemia); ischemic injuries (including myocardial infarction, stroke, and reperfusion injury); and toxin (e.g. alcohol) induced liver disease.

In one aspect, this invention provides isolated nucleic acid molecules encoding programmed cell death-like proteins or biologically active portions thereof, as well as nucleic acid fragments suitable as primers or hybridization probes for the detection of programmed cell death protein like-encoding nucleic acids.

Primary apoptosis deficiencies include graft rejection. Accordingly, the invention is relevant to the identification of genes useful in inhibiting graft rejection.

Primary apoptosis deficiencies also include autoimmune diabetes. Accordingly, the invention is relevant to the identification of genes involved in autoimmune diabetes and accordingly, to the identification of agents that act on these targets to modulate the expression of these genes and hence, to treat or diagnose this disorder. Further, it has been suggested that all auto immune disorders can be viewed as primary deficiencies of apoptosis (Hetts (1998) JAMA 279:300-307). Accordingly, the invention is relevant for screening for gene expression and transcriptional profiling in any autoimmune disorder and for screening for agents that affect the expression or transcriptional profile of these genes.

Primary apoptosis deficiencies also include local self reactive disorder (including Hashimoto thyroiditis), lymphoproliferation, and autoimmunity (including, but not limited to, Canale-Smith syndrome).

Primary apoptosis deficiencies also include cancer. For example, p53 induces apoptosis by acting as a transcription factor that activates expression of various apoptosis-mediating genes or by upregulating apoptosis-mediating genes such as BAX. Other apoptosis-related cancer include, but are not limited to, follicular lymphomas and hormone dependent tumors (including breast, prostate, and ovarian cancer).

Primary apoptosis excesses are associated with neurodegenerative disorders including Alzheimer's disease, Parkinson's disease, spinal muscular atrophy, and amyotrophic lateral sclerosis.

Primary apoptosis excesses are also associated with heart disease including idiopathic dilated cardiomyopathy, ischemic cardiomyopathy, and valvular heart disease. Evidence has also been shown of apoptosis in heart failure resulting from arrhythmogenic right ventricular dysplasia. For all these disorders, see Hetts, above.

A wide variety of neurological diseases are characterized by the gradual loss of specific sets of neurons. Such disorders include Alzheimer's disease, Parkinson's disease, amyotrophic lateral sclerosis (ALS) retinitis pigmentosa, spinal muscular atrophy, and various forms of cerebellar degeneration. The cell loss in these diseases does not induce an inflammatory response, and apoptosis appears to be the mechanism of cell death.

In addition, a number of hematologic diseases are associated with a decreased production of blood cells. These disorders include anemia associated with chronic disease, aplastic anemia, chronic neutropenia, and the myelodysplastic syndromes. Disorders of blood cell production, such as myelodysplastic syndrome and some forms of aplastic anemia, are associated with increased apoptotic cell death within the bone marrow.

These disorders could result from the activation of genes that promote apoptosis, acquired deficiencies in stromal cells or hematopoietic survival factors, or the direct effects of toxins and mediators of immune responses.

Two common disorders associated with cell death are myocardial infarctions and stroke. In both disorders, cells within the central area of ischemia, which is produced in the event of acute loss of blood flow, appear to die rapidly as a result of necrosis. However, outside the central ischemic zone, cells die over a more protracted time period and morphologically appear to die by apoptosis.

The invention also pertains to disorders of the central nervous system (CNS). These disorders include, but are not limited to cognitive and neurodegenerative disorders such as senile dementia, Huntington's disease and Gilles de la Tourette's syndrome, autonomic function disorders such as hypertension and sleep disorders, and neuropsychiatric disorders that include, but are not limited to schizophrenia, schizoaffective disorder, attention deficit disorder, dysthymic disorder, major depressive disorder, mania, obsessive-compulsive disorder, psychoactive substance use disorders, anxiety, panic disorder, as well as bipolar affective disorder, e.g., severe bipolar affective (mood) disorder (BP-I), bipolar affective (mood) disorder with hypomania and major depression (BP-II). Further CNS-related disorders include, for example, those listed in the American Psychiatric Association's Diagnostic and Statistical manual of Mental Disorders (DSM), the most current version of which is incorporated herein by reference in its entirety.

As used herein, “differential expression” or “differentially expressed” includes both quantitative and qualitative differences in the temporal and/or cellular expression pattern of a gene, e.g., the programmed cell death genes disclosed herein, among, for example, normal cells and cells undergoing programmed cell death. Genes which are differentially expressed can be used as part of a prognostic or diagnostic marker for the evaluation of subjects at risk for developing a disorder characterized by deregulated programmed cell death. Depending on the expression level of the gene, the progression state of the disorder can also be evaluated.

Programmed cell death in rat cerebellar granule neurons induced by potassium (K⁺) withdraw has been shown to depend on de novo RNA synthesis. This transcriptional component of CGN programmed cell death was characterized using a custom-built brain-biased cDNA array representing over 7000 different rat genes. Consistent with carefully orchestrated mRNA regulation, the profiles of 234 differentially expressed genes segregated into distinct temporal groups (immediate early, early, middle, and late) encompassing genes involved in distinct physiological responses, including cell-cell signaling, nuclear reorganization, apoptosis, and differentiation. A set of 64 genes, including 22 novel genes, were regulated by both K⁺ withdrawal and kainate treatment. Thus, by using array technology, physiological responses at the transcriptional level were characterized and novel genes induced by multiple models of programmed cell were identified. NARC10 and NARC16 were among these genes.

NARC10 encodes an approximately 2 kb mRNA transcript having the corresponding cDNA set forth in SEQ ID NO: 2. This transcript has a 549 nucleotide open reading frame (nucleotides 95-643 of SEQ ID NO: 2), which encodes a 182 amino acid protein (SEQ ID NO: 1). An analysis of the full-length NARC10 polypeptide using the PSORT Protein Localization algorithm predicts a nuclear localization. Prosite program analysis was used to predict various sites within the NARC10 protein. A protein kinase C phosphorylation site was predicted at amino acid 76-78. A casein kinase II phosphorylation site was predicted at amino acid 57-60. N-myristoylation sites were predicted at amino acid 30-35 and 129-134. The NARC10 protein possesses a nucleosome assembly protein domain (amino acid 78-182) and DNA gyrase/topoisomerase IV, subunit A domain (amino acid 92-110) as predicted by HMMer, Version 2.1.1. Screening the NARC10 protein against the ProDom 2000.1 database revealed that the segment of the protein from amino acid 71-128 contained a nucleosome assembly protein 1-like domain, and the overlapping segment extending from amino acid 68-114 scored as similar the C. elegans hypothetical CAEEL protein which is a putative nucleosome assembly protein. Another overlapping segment, amino acid 55-119, scored as similar to a 1-phosphatidylinositol-4,5bisphosphate phosphodiesterase.

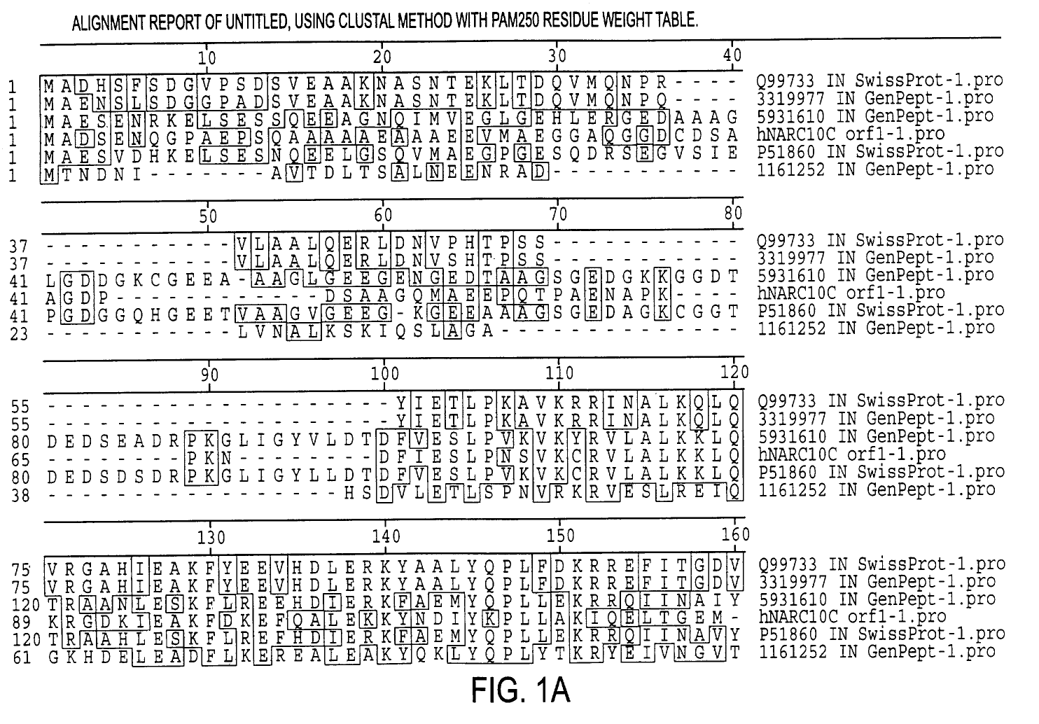

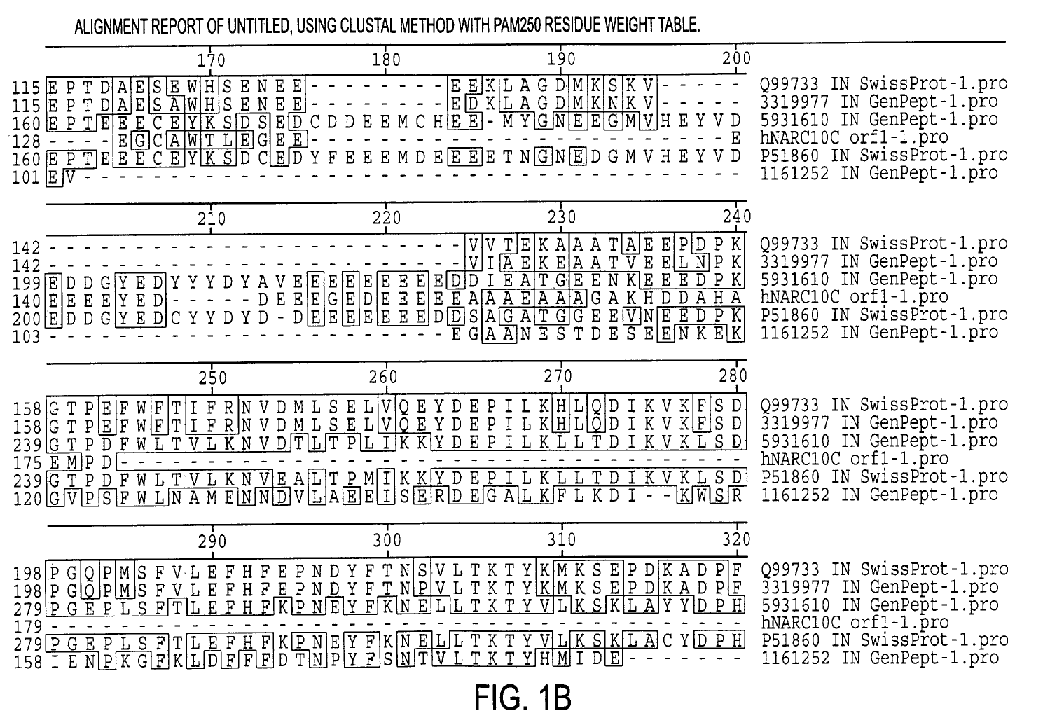

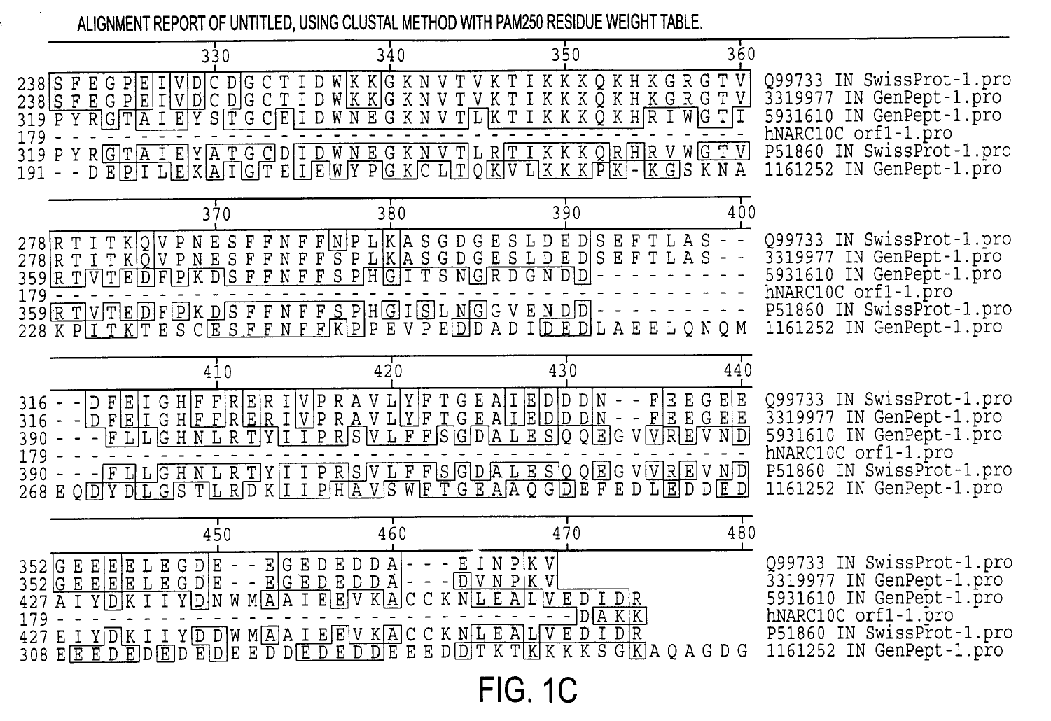

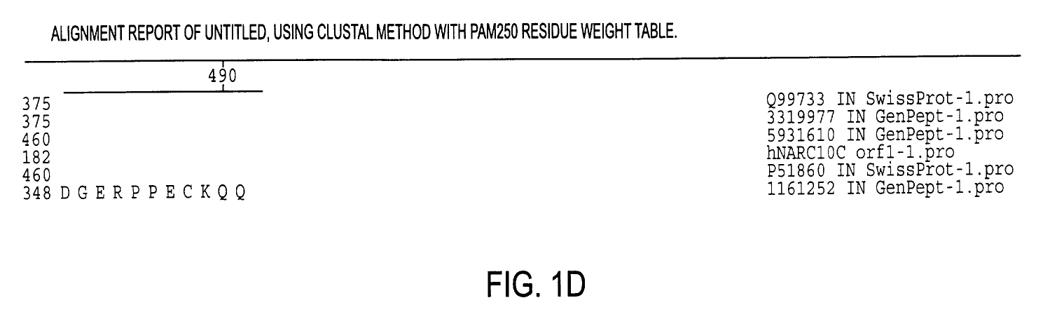

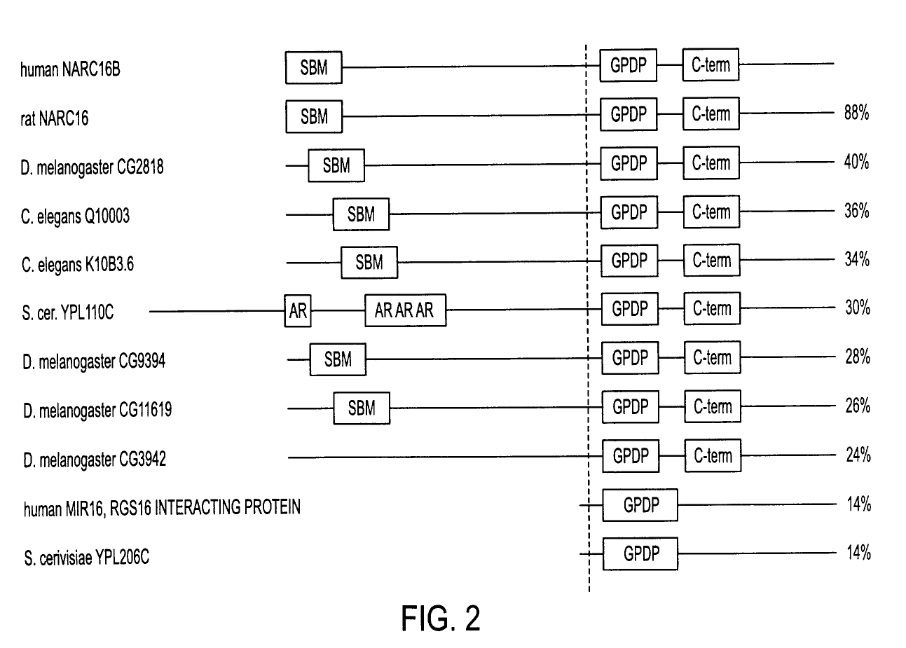

The NARC10 protein displays similarity to human nucleosome protein 1-like protein 4 (SEQ ID NO: 5), murine nucleosome protein 1-like protein 4 (SEQ ID NO: 6), human nucleosome assembly protein 2 (SEQ ID NO: 7), murine nucleosome assembly protein 1-like 2 (SEQ ID NO: 8), and soybean nucleosome assembly protein-1 (SEQ ID NO: 9) (see FIG. 1).

Nucleosome assembly protein-1 (NAP-1) was originally isolated as a histone-binding protein that assembles nucleosome-like structures on a non-replicating DNA template in vitro (Ishimi et al. (1984) Eur. J. Biochem. 142:431-439). Genes encoding NAP-1 are evolutionarily conserved and have been cloned from humans (Simon et al. (1994) Biochem. J. 297:389-397), yeast (Ishimi et al. (1991) J. Biol. Chem. 266:7025-7029), Drosophila (Ito et al. (1996) Mol. Cell. Biol. 16:3112-3124), and Xenopus (Kellogg et al. (1995) J. Cell Biol. 130:661-673). Evidence suggests that NAP-1 functions in nucleosome assembly by serving as a chaperone in the deposition of histones H2A/H2B (Chang et al. (1997) Biochemistry 36:469-480; Bulger et al. (1995) Proc. Natl. Acad. Sci. USA 92:11726-11730). Nap-I from Xenopus and yeast has also been shown to bind to Cyclin B and to be required for Cyclin-B mediated mitotic events (Kellogg et al. (1995) J. Cell. Biol. 130:661-673) This role in the cell cycle may be mediated in part by the Gin4p kinase (Altman et al. (1997) J. Cell. Biol. 138:119-130). Localization studies in Drosophila have demonstrated that Nap-I is primarily nuclear during S-phase of the cell cycle and primarily cytoplasmic during M-phase, consistent with multiple roles for this protein throughout the cell cycle (Ito et al. (1996) Mol. Cell. Biol. 16:3112-3124).

Comparison of the NARC10 nucleotide sequence with the mapped EST database using BLASTN indicated that this gene maps on chromosome 4 at 4q11-4q21. Human diseases that have been shown to map to this area of chromosome 4 include scleroatrophic and keratotic dernatosis of limbs (Huriez Syndrome) and hyper-IgE syndrome.

NARC16 encodes an approximately 3.2 kb mRNA transcript having the corresponding cDNA set forth in SEQ ID NO: 4. This transcript has a 2019 nucleotide open reading frame (nucleotides 145-2163 of SEQ ID NO: 4), which encodes a 672 amino acid protein (SEQ ID NO: 3). An analysis of the full-length NARC16 polypeptide using the PSORT Protein Localization algorithm predicts a cytoplasmic localization. Prosite program analysis was used to predict various sites within the NARC16 protein. N-glycosylation sites were predicted at amino acid 44-47, 328-331, and 472-475. A cAMP and cGMP-dependent protein kinase phosphorylation site was predicted at amino acid 421-424. Protein kinase C phosphorylation sites were predicted at amino acid 140-142, 148-150, 265-267, 281-283, 345-347, 380-382, 440-442, and 494-496. Casein kinase II phosphorylation sites were predicted at amino acid 100-103, 192-195, 201-204, 261-264, 431-434, 447-450, 475-478, 489-492, and 502-505. N-myristoylation sites were predicted at amino acid 24-29, 114-119, 325-330, and 467-472. An amidation site was predicted for amino acid 494-497. The NARC16 protein possesses a starch biding domain (amino acid 3-110) as predicted by HMMer, Version 2.1.1. ProDom analysis indicated that NARC16 contains a glycerophosphoryl diester glycerophosphodiesterase domain (aa 321-374), and a glycerophosphoryl diester phosphodiesterase protein T05H10.7-like domain (amino acid 22-138, 270-316, and 574-595). Procaryotic glycerophosphoryl diester glycerophosphodiesterase is a dimeric periplasmically-located enzyme that hydrolyzes deicetylated phospholipids to produce glycerol 3-phosphate and an alcohol (Larson et al. (1983) J. Biol. Chem. 258:5426-5432. Recently, a human protein (MIR 16) with significant similarity to bacterial glycerophosphodiester phosphodiesterase was isolated and is postulated to play a role in lipid metabolism and G protein signaling (Zheng et al. (2000) Proc. Natl. Acad. Sci. USA 97:3999-4004).

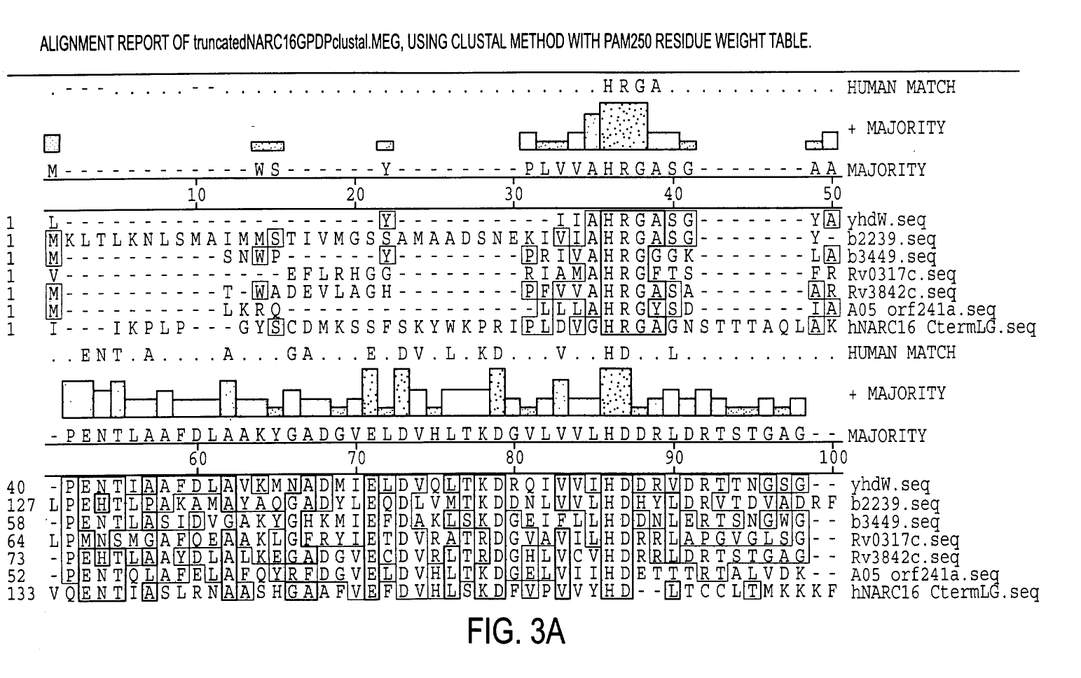

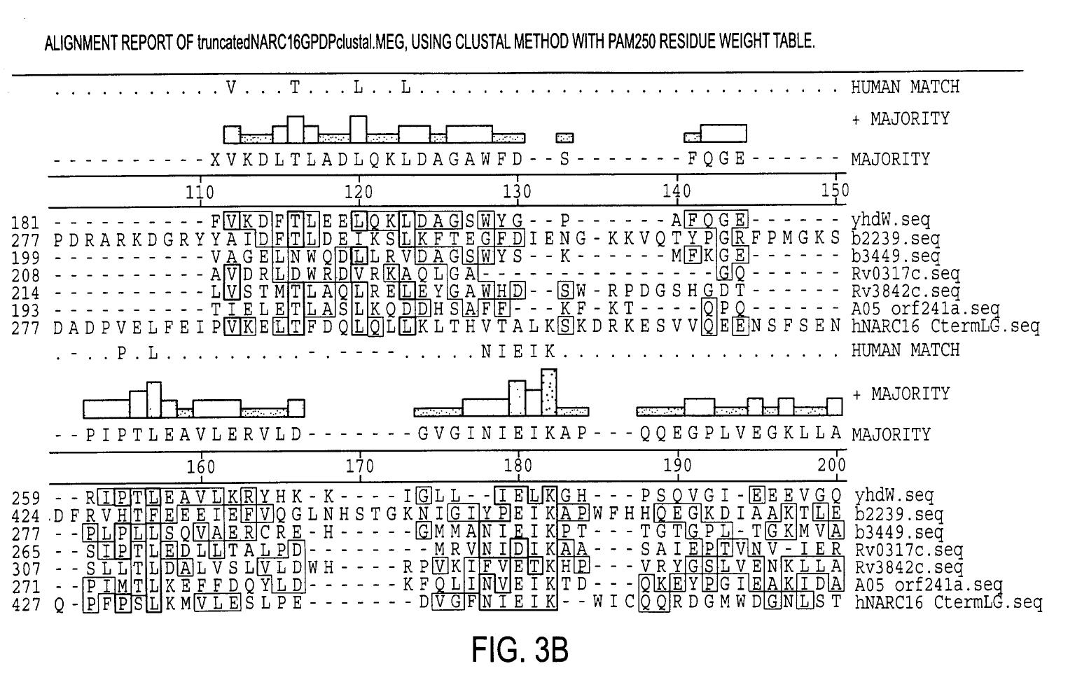

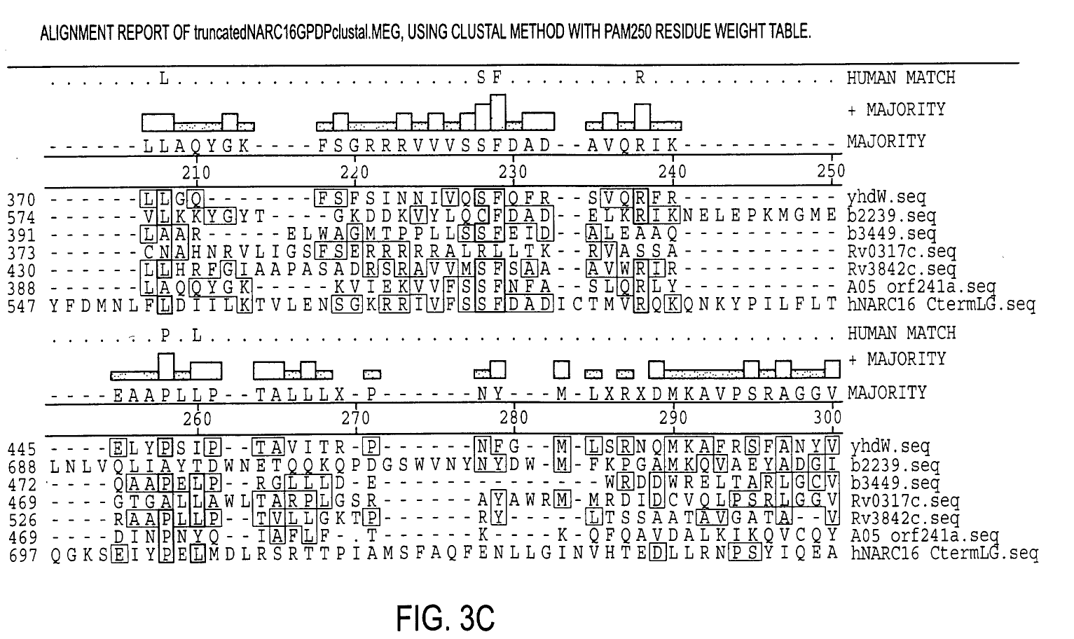

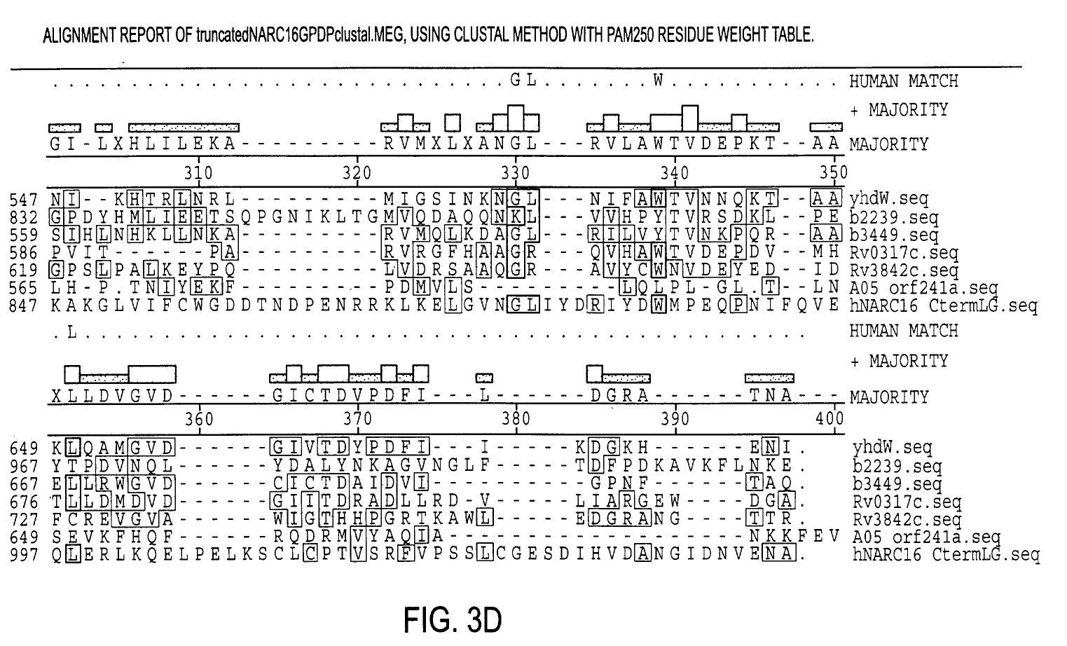

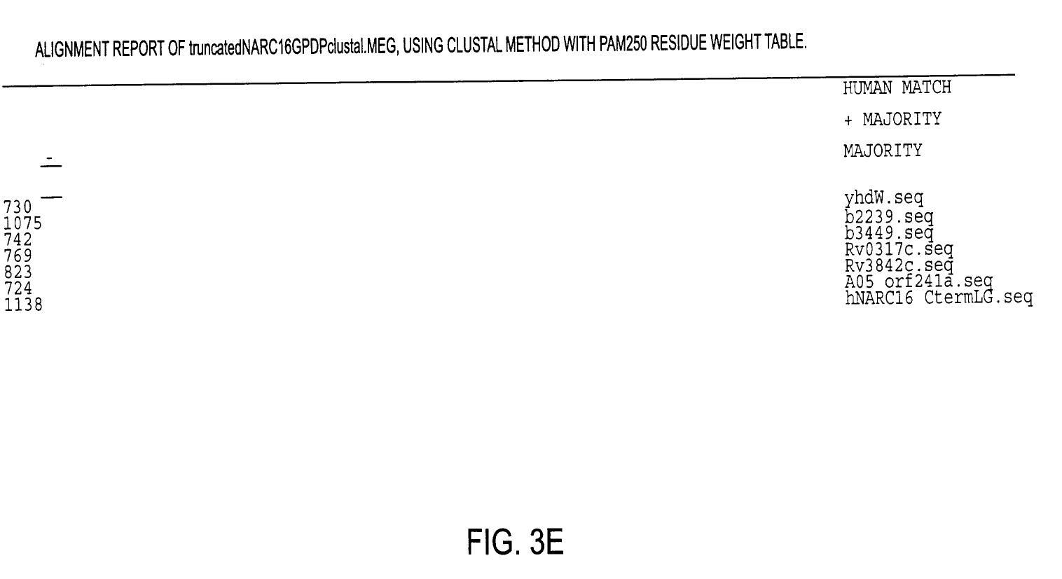

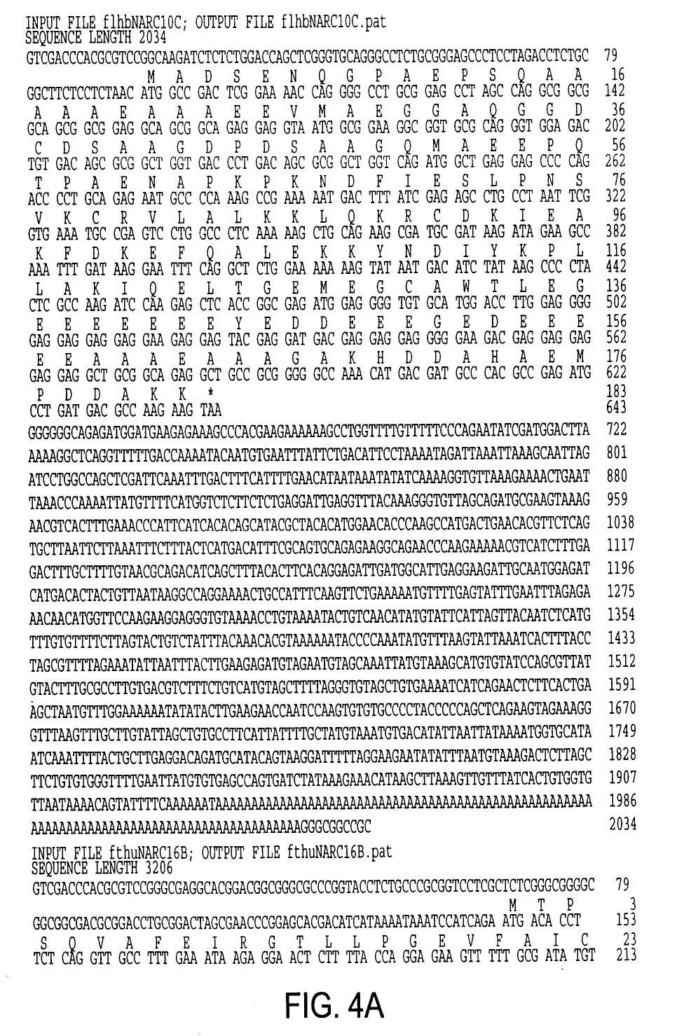

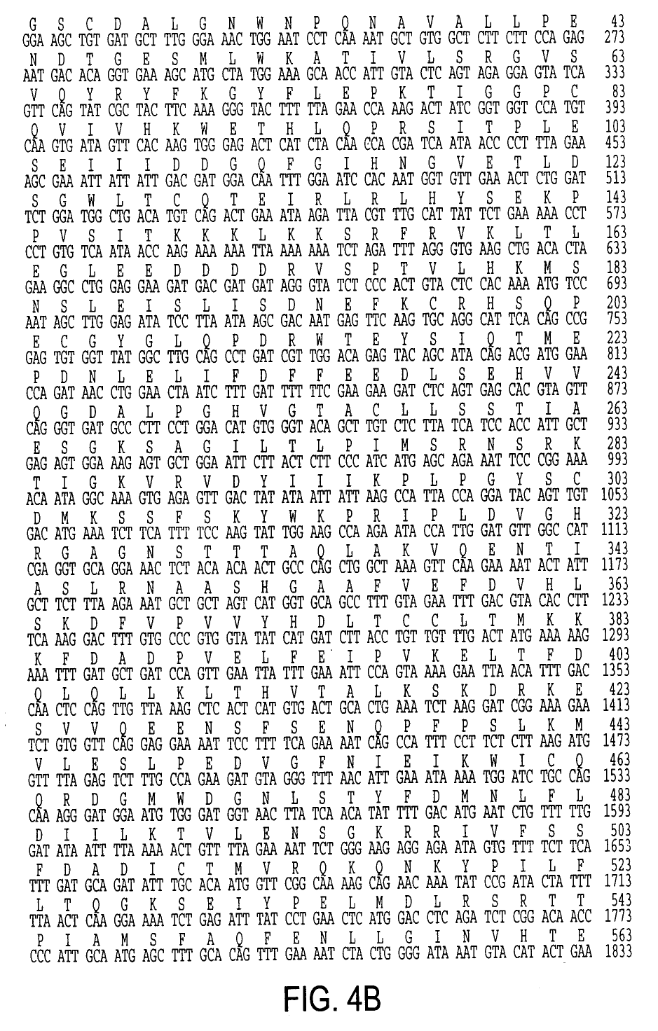

The NARC16 protein shares sequence similarity with Bacillus subtilus glycerophosphoryl phosphodiester phosphodiesterase (SEQ ID NO: 11), Escherichia coli glycerophosphoryl phosphodiester phosphodiesterase (SEQ ID NO: 12), cytosolic Escherichia coli glycerophosphoryl phosphodiester phosphodiesterase (SEQ ID NO: 13), Mycobacterium tuberculosis glycerophosphoryl phosphodiester phosphodiesterase glpQ2 (SEQ ID NO: 14), Mycobacterium tuberculosis glycerophosphoryl phosphodiester phosphodiesterase glpQ1 (SEQ ID NO: 15), and Mycoplasma pneumoniae glycerophosphoryl phosphodiester phosphodiesterase (SEQ ID NO: 16)(see FIG. 3).

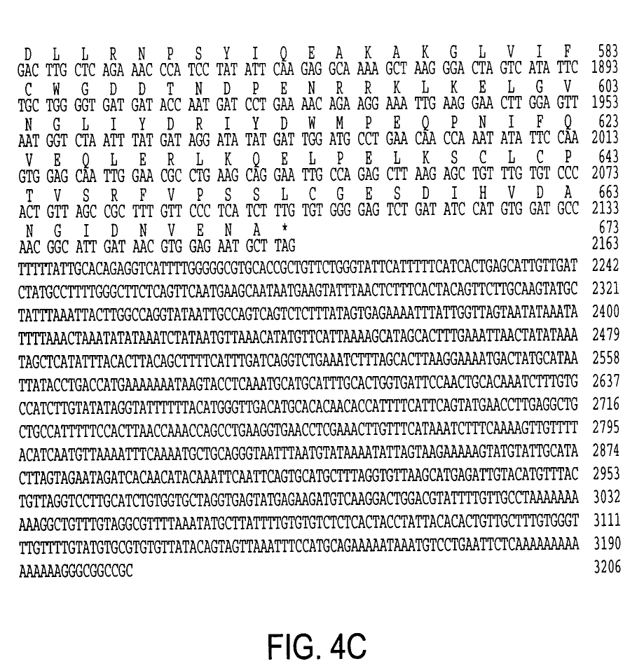

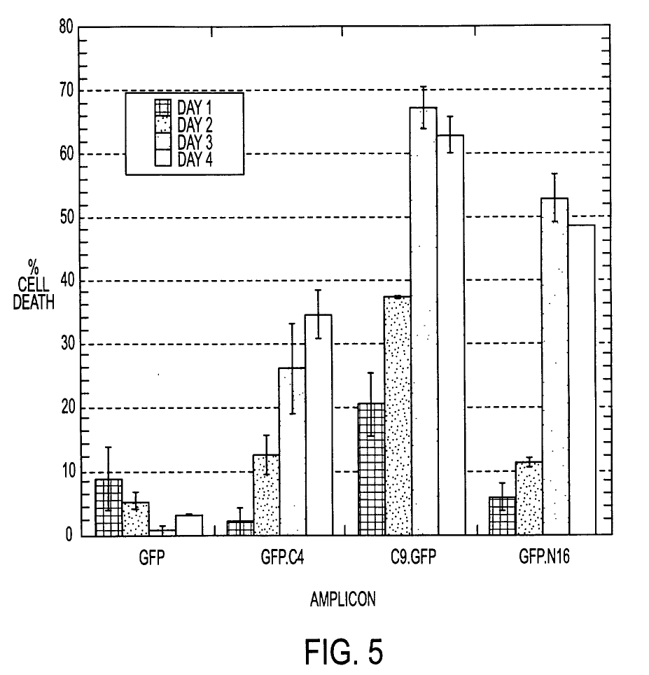

Overexpression of NARC16 kills cerebellar granule neurons (see FIG. 5), PC12 cells, and rat-1 cells. The apoptotic effects of NARC16 in rat-1 cells can be rescued by BCl-X_{L} (which has been shown to protect cells from cell death) and caspase inhibitors. The present invention is not held to any particular mechanism of for the action of NARC16 in promoting apoptosis. It is, however, believed that NARC16 may promote programmed cell death via its glycerophosphoryl phosphodiesterase activity.

Glycerophosphoryl phosphodiesterases are involved in the breakdown of phospholipids. Fatty acids are removed from phospholipids (e.g. phosphotidyl choline, phosphotidyl inositol, phosphotidyl ethanolamine, phosphotidyl glycerol, or phosphotidyl serine) via phospholipase A1 and A2 lysophospholipase to form glycerol phosphoryl diesters (e.g. glycerophosphocholine, glycerophosphoinositol, glycerophosphoethanolamine, glycerophosphoglycerol, or glycerophosphoserine). These glycerol phosphoryl diesters are then hydrolyzed by glycerophosphoryl phosphodiesterase to form glycerol-3-phosphate and various alcohols.

Glycerol-3-phosphate, the hydrolysis product of glycerophosphoryl phosphodiester phosphodiesterases, is a precursor in the formation of glyceraldehyde 3-phosphate. Glycerol-3-phosphate is converted to glycerone-P (dihydroxyacetone phosphate) via glycerol 3-phosphate dehydrogenase, and glycerone-P is converted to glyceraldehyde 3-phosphate by triose phosphate isomerase. Glyceraldehyde-3-phosphate is the substrate of the glytolytic enzyme glyceraldehyde 3-phosphate dehydrogenase (GAPDH), and this enzyme has been implicated as a general mediator of programmed cell death.

The role of GAPDH in apoptosis was first demonstrated when it was observed that the appearance of this protein coincided with the induction of apoptosis in cerebral granular cells (reviewed in Sirover (1999) Biochim. Biophys. Acta 1432:159-184). The physiological relevance of GAPDH biosynthesis in neuronal apoptosis was further defined by antisense studies, where it was shown that transfection of cerebral granular cells with antisense GAPDH inhibited programmed cell death. It has also been observed that a proposed anti-dementia drug, ONO-1603, which delays apoptosis in neuronal cells, also decreases GAPDH gene expression (reviewed in Sirover, ibid).

GAPDH has been shown to interact specifically with a number of proteins involved in human neuronal disorders, including the β-amyloid precursor protein (involved in Alzheimer's disease), the Huntingtin protein (involved in Huntington's disease), atrophin (involved in dentatorubalpallidoluysian atrophy), ataxin (involved in spinocerebellar ataxia type-1), and the androgen receptor (involved in spinobulbar muscular atrophy) (reviewed by Sirover, ibid). Further evidence for the role of GAPDH in neurodegenerative disease comes from the finding that the anti-apoptotic Parkinson's disease drug R-(−)-deprenyl (selegiline), as well as a related anti-apoptotic compound, CGP 3466, bind specifically to GAPDH (Krageten (1998) J. Biol. Chem. 273:5821-5828). Selegiline has also been shown to alter the expression of genes that influence mitochondrial viability (including superoxide dismutase 1 and 2), as well as the apoptosis-related genes Bcl-2, BCl-X_{L}, and BAX (reviewed in Tatton et al. (1996) Neurology 47(Suppl 3):S171-S183. Accordingly, it is proposed that NARC16 may promote apoptosis by activating the pro-apoptotic activities of GAPDH.

Comparison of the NARC16 nucleotide sequence with the mapped EST database using BLASTN indicated that this gene maps on chromosome 20 at 20p13−20p12. Human diseases that have been shown to map to this area of chromosome 20 include Hallervorden-Spatz Disease (late infantile neuroaxonal dystrophy) and corneal endothelial dystrophy 2 (CHED2).

The programmed cell death-related sequences of the invention are each a member of a family of molecules having conserved functional features. The term “family” when referring to the proteins and nucleic acid molecules of the invention is intended to mean two or more proteins or nucleic acid molecules having sufficient amino acid or nucleotide sequence identity as defined herein. Such family members can be naturally occurring and can be from either the same or different species. For example, a family can contain a first protein of murine origin and a homologue of that protein of human origin, as well as a second, distinct protein of human origin and a murine homologue of that protein. Members of a family may also have common functional characteristics.

Preferred programmed cell death-related polypeptides of the present invention have an amino acid sequence sufficiently identical to the amino acid sequence of SEQ ID NO: 1 or SEQ ID NO: 3. The term “sufficiently identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity. For example, amino acid or nucleotide sequences that contain a common structural domain having at least about 45%, 55%, or 65% identity, preferably 75% identity, more preferably 85%, 95%, or 98% identity are defined herein as sufficiently identical.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. The percent identity between two sequences can be determined using techniques similar to those described below, with or without allowing gaps. In calculating percent identity, typically exact matches are counted.

The determination of percent identity between two sequences can be accomplished using a mathematical algorithm. A nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:403. BLAST nucleotide searches can be performed with the NBLAST program, score=100, wordlength=12, to obtain nucleotide sequences homologous to programmed cell death-like nucleic acid molecules of the invention. BLAST protein searches can be performed with the XBLAST program, score=50, wordlength=3, to obtain amino acid sequences homologous to programmed cell death-like protein molecules of the invention. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST) can be used. See http://www.ncbi.nlm.nih.gov. Another non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

In a preferred embodiment, the percent identity between two amino acid sequences is determined using the Needleman and Wunsch (1970) J. Mol. Biol. 48:444-453 algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) is using a Blossum 62 scoring matrix with a gap open penalty of 12, a gap extend penalty of 4, and a frameshift gap penalty of 5.

Another embodiment of the invention features isolated programmed cell death-related polypeptides having a NARC10 or NARC16 activity, referred to collectively as a “programmed cell death-related polypeptide activity”. As used interchangeably herein, “NARC10 activity”, “NARC16 activity”, “programmed cell death-related polypeptide activity”, “biological activity of a programmed cell death-related protein”, or “functional activity of a programmed cell death-related polypeptide” refers to an activity exerted by a programmed cell death-related polypeptide, polypeptide, or nucleic acid molecule on a programmed cell death-related responsive cell as determined in vivo, or in vitro, according to standard assay techniques. A programmed cell death-related activity can be a direct activity, such as an association with or an enzymatic activity on a second protein, or an indirect activity, such as a cellular signaling activity mediated by interaction of the programmed cell death-related polypeptide with a second protein. For example, a NARC10 activity includes at least one or more of the following activities: (1) modulating programmed cell death or apoptosis; (2) modulating the cell cycle in a NARC10-dependent manner; (3) modulating chromatin assembly. Examples of NARC16 activity include: (1)modulating programmed cell death or apoptosis, (2), modulating the cell cycle in a NARC16-dependent manner, and (3) hydrolysing glycerol phosphoryl diesters to form glycerol-3-phosphate and an alcohol. The modulation of programmed cell death may be assayed by any method known in the art, including the methods described elsewhere herein. Methods of assaying cell cycle progression are also well know in the art and may be used to determine the modulatory effects of a polypeptide on the cell cycle. Chromatin assembly may be assayed by any method known in the art, including the methods described in Ishimi et al. (1984), Eur. J. Biochem. 142:431-9, and McQuibban et al. (1998), J. Biol. Chem. 273:6582-6590; herein incorporated by reference. The hydrolysis of glycerol phsophoryl diesters may be assayed by any method known in the art, including the methods described in Larson et al. (1983) J. Biol. Chem. 258:54t28-5432, Paltauf et al. (1985) Biochim. Biophys. Acta 835:322-330, Brzoska and Boos (1988) J. Bacteriol. 170:4125-4135, Hawkins et al. (1993) J. Biol. Chem. 268:3374-3383, and Marino et al. (1996) Eur. J. Biochem. 241:386-392; herein incorporated by reference.

An “isolated” or “purified” programmed cell death protein-like nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5N and 3N ends of the nucleic acid) in the genomic DNA of the organism from which the nucleic acid is derived. For purposes of the invention, “isolated” when used to refer to nucleic acid molecules excludes isolated chromosomes. For example, in various embodiments, the programmed cell death-related nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A programmed cell death-related polypeptide that is substantially free of cellular material includes preparations of programmed cell death-related polypeptide having less than about 30%, 20%, 10%, or 5% (by dry weight) of non-programmed cell death-related polypeptide (also referred to herein as a “contaminating protein”). When the programmed cell death-related polypeptide or biologically active portion thereof is recombinantly produced, preferably, culture medium represents less than about 30%, 20%, 10%, or 5% of the volume of the protein preparation. When programmed cell death-related polypeptide is produced by chemical synthesis, preferably the protein preparations have less than about 30%, 20%, 10%, or 5% (by dry weight) of chemical precursors or non-programmed cell death-related polypeptide chemicals.

Various aspects of the invention are described in further detail in the following subsections.

I. Isolated Nucleic Acid Molecules

One aspect of the invention pertains to isolated nucleic acid molecules comprising nucleotide sequences encoding programmed cell death-related polypeptides and polypeptides or biologically active portions thereof, as well as nucleic acid molecules sufficient for use as hybridization probes to identify programmed cell death related polypeptide encoding nucleic acids (e.g., programmed cell death related mRNA) and fragments for use as PCR primers for the amplification or mutation of programmed cell death-related nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

Nucleotide sequence encoding the programmed cell death-related polypeptides of the present invention include sequences set forth in SEQ ID NO: 2 and SEQ ID NO: 4, and complements thereof. By “complement” is intended a nucleotide sequence that is fully complementary to a given nucleotide sequence. The corresponding amino acid sequence for the programmed cell death-related polypeptides encoded by these nucleotide sequences is set forth in SEQ ID NO: 1 and SEQ ID NO: 3.

Nucleic acid molecules that are fragments of programmed cell death-related nucleotide sequences are also encompassed by the present invention. By “fragment” is intended a portion of the nucleotide sequence encoding a programmed cell death-related polypeptide. A fragment of programmed cell death-related nucleotide sequence may encode a biologically active portion of a programmed cell death-related polypeptide, or it may be a fragment that can be used as a hybridization probe or PCR primer using methods disclosed below. A biologically active portion of a programmed cell death-related polypeptide can be prepared by isolating a portion of one of the NARC10 or NARC16 nucleotide sequences of the invention, expressing the encoded portion of the programmed cell death-related polypeptide (e.g., by recombinant expression in vitro), and assessing the activity of the encoded portion of the programmed cell death-related polypeptide. Nucleic acid molecules that are fragments of programmed cell death-related nucleotide sequence comprise at least about 15, 20, 50, 75, 100, 200, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, or 3200 nucleotides, or up to the number of nucleotides present in a full-length programmed cell death-related nucleotide sequence disclosed herein (for example, 2034 nucleotides for SEQ ID NO: 2 or 3206 nucleotides for SEQ ID NO: 4) depending upon the intended use.

It is understood that isolated fragments include any contiguous sequence not disclosed prior to the invention as well as sequences that are substantially the same and which are not disclosed. Accordingly, if an isolated fragment is disclosed prior to the present invention, that fragment is not intended to be encompassed by the invention. When a sequence is not disclosed prior to the present invention, an isolated nucleic acid fragment is at least about 12, 15, 20, 25, or 30 contiguous nucleotides. Other regions of the nucleotide sequence may comprise fragments of various sizes, depending upon potential homology with previously disclosed sequences.

A fragment of a programmed cell death-related nucleotide sequence that encodes a biologically active portion of a programmed cell death-related polypeptide of the invention will encode at least about 15, 25, 30, 50, 75, 100, 125, 150, 175, 200, 250, 300, 350, 400, 450, 500, 550, 600, or 650 contiguous amino acids, or up to the total number of amino acids present in a full-length programmed cell death-related polypeptide of the invention (for example, about 182 amino acids for SEQ ID NO: 1, or about 672 amino acids for SEQ ID NO: 3). Fragments of a programmed cell death-related nucleotide sequence that are useful as hybridization probes for PCR primers generally need not encode a biologically active portion of a programmed cell death-related polypeptide.

Nucleic acid molecules that are variants of the programmed cell death-related nucleotide sequences disclosed herein are also encompassed by the present invention. “Variants” of the programmed cell death-related nucleotide sequences include those sequences that encode the programmed cell death-related polypeptides disclosed herein but that differ conservatively because of the degeneracy of the genetic code. These naturally occurring allelic variants can be identified with the use of well-known molecular biology techniques, such as polymerase chain reaction (PCR) and hybridization techniques as outlined below. Variant nucleotide sequences also include synthetically derived nucleotide sequences that have been generated, for example, by using site-directed mutagenesis but which still encode the programmed cell death-related polypeptides disclosed in the, present invention as discussed below. Generally, nucleotide sequence variants of the invention will have at least about 45%, 55%, 65%, 75%, 85%, 95%, or 98% identity to a particular nucleotide sequence (SEQ ID NO: 2 or SEQ ID NO: 4) disclosed herein, and the polypeptide encoded by the nucleotide sequence variant will retain the activity of the polypeptide encoded by the reference sequence. A variant programmed cell death-related nucleotide sequence will encode a programmed cell death-related polypeptide that has an amino acid sequence having at least about 45%, 55%, 65%, 75%, 85%, 95%, or 98% identity to the amino acid sequence of a programmed cell death-related polypeptide disclosed herein (e.g. SEQ ID NO: 1 or SEQ ID NO: 3).

In addition to the programmed cell death-related nucleotide sequences shown in SEQ ID NO: 2 and SEQ ID NO: 4 respectively, it will be appreciated by those skilled in the art that DNA sequence polymorphisms that lead to changes in the amino acid sequences of programmed cell death protein-like proteins may exist within a population (e.g., the human population). Such genetic polymorphism in a programmed cell death-related gene may exist among individuals within a population due to natural allelic variation. An allele is one of a group of genes that occur alternatively at a given genetic locus. As used herein, the terms “gene” and “recombinant gene” refer to nucleic acid molecules comprising an open reading frame encoding a programmed cell death-related polypeptide, preferably a mammalian programmed cell death-related polypeptide. As used herein, the phrase “allelic variant” refers to a nucleotide sequence that occurs at a programmed cell death-related locus or to a polypeptide encoded by the nucleotide sequence. Such natural allelic variations can typically result in 1-5% variance in the nucleotide sequence of the programmed cell death-related gene. Any and all such nucleotide variations and resulting amino acid polymorphisms or variations in programmed cell death-related sequence that are the result of natural allelic variation and that do not alter the functional activity of programmed cell death-related polypeptides are intended to be within the scope of the invention.

Moreover, nucleic acid molecules programmed cell death-related polypeptides from other species (programmed cell death-related homologues), which have a nucleotide sequence differing from that of the programmed cell death-related sequences disclosed herein, are intended to be within the scope of the invention. For example, nucleic acid molecules corresponding to natural allelic variants and homologues of the human programmed cell death-related cDNA of the invention can be isolated based on their identity to the human programmed cell death-related nucleic acid disclosed herein using the human cDNA, or a portion thereof, as a hybridization probe according to standard hybridization techniques under stringent hybridization conditions as disclosed below.

In addition to naturally-occurring allelic variants of the programmed cell death-related sequences that may exist in the population, the skilled artisan will further appreciate that changes can be introduced by mutation into the nucleotide sequences of the invention thereby leading to changes in the amino acid sequence of the encoded programmed cell death-related polypeptides, without altering the biological activity of the programmed cell death-related polypeptides. Thus, an isolated nucleic acid molecule encoding a programmed cell death-related polypeptide having a sequence that differs from that of SEQ ID NO: 2 or SEQ ID NO: 4, can be created by introducing one or more nucleotide substitutions, additions, or deletions into the corresponding nucleotide sequence disclosed herein, such that one or more amino acid substitutions, additions or deletions are introduced into the encoded protein. Mutations can be introduced by standard techniques, such as site-directed mutagenesis and PCR-mediated mutagenesis. Such variant nucleotide sequences are also encompassed by the present invention.

For example, preferably, conservative amino acid substitutions may be made at one or more predicted, preferably nonessential amino acid residues. A “nonessential” amino acid residue is a residue that can be altered from the wild-type sequence of a programmed cell death-related polypeptide (e.g., the sequences of SEQ ID NO: 1 or SEQ ID NO: 3) without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Such substitutions would not be made for conserved amino acid residues, or for amino acid residues residing within a conserved motif.

Alternatively, variant programmed cell death-related nucleotide sequences can be made by introducing mutations randomly along all or part of a programmed cell death-related coding sequence, such as by saturation mutagenesis, and the resultant mutants can be screened for programmed cell death-related biological activity to identify mutants that retain activity. Following mutagenesis, the encoded protein can be expressed recombinantly, and the activity of the protein can be determined using standard assay techniques.

Thus the nucleotide sequences of the invention include the sequences disclosed herein as well as fragments and variants thereof. The programmed cell death-related nucleotide sequences of the invention, and fragments and variants thereof, can be used as probes and/or primers to identify and/or clone programmed cell death-related homologues in other cell types, e.g., from other tissues, as well as programmed cell death-related homologues from other mammals. Such probes can be used to detect transcripts or genomic sequences encoding the same or identical proteins. These probes can be used as part of a diagnostic test kit for identifying cells or tissues that misexpress a programmed cell death-related polypeptide, such as by measuring levels of a programmed cell death protein-like-encoding nucleic acid in a sample of cells from a subject, e.g., detecting programmed cell death-related mRNA levels or determining whether a genomic programmed cell death-related gene has been mutated or deleted.

In this manner, methods such as PCR, hybridization, and the like can be used to identify such sequences having substantial identity to the sequences of the invention. See, for example, Sambrook et al. (1989) Molecular Cloning: Laboratory Manual (2d ed., Cold Spring Harbor Laboratory Press, Plainview, N.Y.) and Innis, et al. (1990) PCR Protocols: A Guide to Methods and Applications (Academic Press, NY). Programmed cell death-related nucleotide sequences isolated based on their sequence identity to the programmed cell death-related nucleotide sequences set forth herein or to fragments and variants thereof are encompassed by the present invention.

In a hybridization method, all or part of a programmed cell death-related nucleotide sequence can be used to screen cDNA or genomic libraries. Methods for construction of such cDNA and genomic libraries are generally known in the art and are disclosed in Sambrook et al. (1989) Molecular Cloning: A Laboratory Manual (2d ed., Cold Spring Harbor Laboratory Press, Plainview, N.Y.). The so-called hybridization probes may be genomic DNA fragments, cDNA fragments, RNA fragments, or other oligonucleotides, and may be labeled with a detectable group such as ³²P, or any other detectable marker, such as other radioisotopes, a fluorescent compound, an enzyme, or an enzyme co-factor. Probes for hybridization can be made by labeling synthetic oligonucleotides based on the known programmed cell death-related nucleotide sequence disclosed herein. Degenerate primers designed on the basis of conserved nucleotides or amino acid residues in a known programmed cell death-related nucleotide sequence or encoded amino acid sequence can additionally be used. The probe typically comprises a region of nucleotide sequence that hybridizes under stringent conditions to at least about 12, preferably about 25, more preferably about 50, 75, 100, 125, 150, 175, 200, 250, 300, 350, or 400 consecutive nucleotides of a programmed cell death protein-like nucleotide sequence of the invention or a fragment or variant thereof. Preparation of probes for hybridization is generally known in the art and is disclosed in Sambrook et al. (1989) Molecular Cloning: A Laboratory Manual (2d ed., Cold Spring Harbor Laboratory Press, Plainview, N.Y.), herein incorporated by reference.

For example, in one embodiment, a previously unidentified NARC10-like programmed cell death-related molecule hybridizes under stringent conditions to a probe that is a nucleic acid molecule comprising one of the NARC10 nucleotide sequences of the invention or a fragment thereof. In another embodiment, the previously unknown NARC10-like programmed cell death-related nucleic acid molecule is at least about 300, 325, 350, 375, 400, 425, 450, 500, 550, 600, 650, 700, 800, 900, 1000, 2,000, or 2034 nucleotides in length and hybridizes under stringent conditions to a probe that is a nucleic acid molecule comprising one of the NARC10 nucleotide sequences of the invention, preferably the coding sequence set forth in SEQ ID NO: 4 or a complement thereof.

Accordingly, in another embodiment, an isolated previously unknown NARC16-like programmed cell death-related nucleic acid molecule of the invention is at least about 300, 325, 350, 375, 400, 425, 450, 500, 550, 600, 650, 700, 800, 900, 1000, 1,100, 1,200, 1,300, 1,400, 1500, 2000, 2500, 3000, or 3206 nucleotides in length and hybridizes under stringent conditions to a probe that is a nucleic acid molecule comprising one of the NARC16 nucleotide sequences of the invention, preferably the coding sequence set forth in SEQ ID NO: 2 or a complement, fragment, or variant thereof.

As used herein, the term “hybridizes under stringent conditions” is intended to describe conditions for hybridization and washing under which nucleotide sequences having at least about 60%, 65%, 70%, preferably 75% identity to each other typically remain hybridized to each other. Such stringent conditions are known to those skilled in the art and can be found in Current Protocols in Molecular Biology (John Wiley & Sons, New York (1989)), 6.3.1-6.3.6. One preferred example of stringent hybridization conditions comprises hybridization in 6× sodium chloride/sodium citrate (SSC) at about 45° C., followed by one or more washes in 0.2× SSC, 0.1% SDS at 50-65° C. Another preferred example of stringent conditions comprises hybridization in 6× SSC at 42° C., followed by washing with 1× SSC at 55° C. Particularly preferred stringency conditions (and the conditions should be used if the practitioner is uncertain about what conditions should be applied to determine if a molecule is within a hybridization limitation of the invention) comprise 0.5 M sodium phosphate, 7% SDS for 16 hours, followed by at least one wash at 0.2× SSC, 1% SDS at 65° C. for at least 15 minutes. Preferably, an isolated nucleic acid molecule that hybridizes under stringent conditions to a programmed cell death-like sequence of the invention corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

Thus, in addition to the programmed cell death-like nucleotide sequences disclosed herein and fragments and variants thereof, the isolated nucleic acid molecules of the invention also encompass homologous DNA sequences identified and isolated from other cells and/or organisms by hybridization with entire or partial sequences obtained from the programmed cell death-related nucleotide sequences disclosed herein or variants and fragments thereof.

The present invention also encompasses antisense nucleic acid molecules, i.e., molecules that are complementary to a sense nucleic acid encoding a protein, e.g., complementary to the coding strand of a double-stranded cDNA molecule, or complementary to an mRNA sequence. Accordingly, an antisense nucleic acid can hydrogen bond to a sense nucleic acid. The antisense nucleic acid can be complementary to an entire the programmed cell death-like coding strand, or to only a portion thereof, e.g., all or part of the protein coding region (or open reading frame). An antisense nucleic acid molecule can be antisense to a noncoding region of the coding strand of a nucleotide sequence encoding an the programmed cell death-related polypeptide. The noncoding regions are the 5′ and 3′ sequences that flank the coding region and are not translated into amino acids.

Given the coding-strand sequence encoding an the programmed cell death-related polypeptide disclosed herein (e.g., SEQ ID NO: 2 and SEQ ID NO: 4), antisense nucleic acids of the invention can be designed according to the rules of Watson and Crick base pairing. The antisense nucleic acid molecule can be complementary to the entire coding region of the programmed cell death-related mRNA, but more preferably is an oligonucleotide that is antisense to only a portion of the coding or noncoding region of programmed cell death-related mRNA. For example, the antisense oligonucleotide can be complementary to the region surrounding the translation start site of programmed cell death-related mRNA. An antisense oligonucleotide can be, for example, about 5, 10, 15, 20, 25, 30, 35, 40, 45, or 50 nucleotides in length. An antisense nucleic acid of the invention can be constructed using chemical synthesis and enzymatic ligation procedures known in the art.

For example, an anti sense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, including, but not limited to, for example e.g., phosphorothioate derivatives and acridine substituted nucleotides. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

When used therapeutically, the antisense nucleic acid molecules of the invention are typically administered to a subject or generated in situ such that they hybridize with or bind to cellular mRNA and/or genomic DNA encoding a programmed cell death protein-like protein to thereby inhibit expression of the protein, e.g., by inhibiting transcription and/or translation. An example of a route of administration of antisense nucleic acid molecules of the invention includes direct injection at a tissue site. Alternatively, antisense nucleic acid molecules can be modified to target selected cells and then administered systemically. For example, antisense molecules can be linked to peptides or antibodies to form a complex that specifically binds to receptors or antigens expressed on a selected cell surface. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

The invention also encompasses ribozymes, which are catalytic RNA molecules with ribonuclease activity that are capable of cleaving a single-stranded nucleic acid, such as an mRNA, to which they have a complementary region. Ribozymes (e.g., hammerhead ribozymes (described in Haselhoff and Gerlach (1988) Nature 334:585-591)) can be used to catalytically cleave programmed cell death-related mRNA transcripts to thereby inhibit translation of programmed cell death-related mRNA. A ribozyme having specificity for a programmed cell death protein-like-encoding nucleic acid can be designed based upon the nucleotide sequence of a programmed cell death-related cDNA disclosed herein (e.g., SEQ ID NO: 2 or SEQ ID NO: 4). See, e.g., Cech et al., U.S. Pat. No. 4,987,071; and Cech et al., U.S. Pat. No. 5,116,742. Alternatively, programmed cell death-related mRNA can be used to select a catalytic RNA having a specific ribonuclease activity from a pool of RNA molecules. See, e.g., Bartel and Szostak (1993) Science 261:1411-1418.

The invention also encompasses nucleic acid molecules that form triple helical structures. For example, programmed cell death-related gene expression can be inhibited by targeting nucleotide sequences complementary to the regulatory region of the programmed cell death-related polypeptide (e.g., the programmed cell death-related promoter and/or enhancers) to form triple helical structures that prevent transcription of the programmed cell death-related gene in target cells. See, generally Helene (1991) Anticancer Drug Des. 6(6):569; Helene (1992) Ann. N.Y. Acad. Sci. 660:27; and Maher (1992) Bioassays 14(12):807.

In preferred embodiments, the nucleic acid molecules of the invention can be modified at the base moiety, sugar moiety, or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4:5). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid-phase peptide synthesis protocols as described, for example, in Hyrup et al. (1996), supra; Perry-O'Keefe et al. (1996) Proc. Natl. Acad. Sci. USA 93:14670.

PNAs of a programmed cell death-related molecule can be used in therapeutic and diagnostic applications. For example, PNAs can be used as antisense or antigene agents for sequence-specific modulation of gene expression by, e.g., inducing transcription or translation arrest or inhibiting replication. PNAs of the invention can also be used, e.g., in the analysis of single base pair mutations in a gene by, e.g., PNA-directed PCR clamping; as artificial restriction enzymes when used in combination with other enzymes, e.g., S1 nucleases (Hyrup (1996), supra); or as probes or primers for DNA sequence and hybridization (Hyrup (1996), supra; Perry-O'Keefe et al. (1996), supra).

In another embodiment, PNAs of a programmed cell death-related molecule can be modified, e.g., to enhance their stability, specificity, or cellular uptake, by attaching lipophilic or other helper groups to PNA, by the formation of PNA-DNA chimeras, or by the use of liposomes or other techniques of drug delivery known in the art. The synthesis of PNA-DNA chimeras can be performed as described in Hyrup (1996), supra; Finn et al. (1996) Nucleic Acids Res. 24(17):3357-63; Mag et al. (1989) Nucleic Acids Res. 17:5973; and Peterson et al. (1975) Bioorganic Med. Chem. Lett. 5:1119.

II. Isolated Programmed Cell Death-Related Polypeptides and Antibodies

Programmed cell death-related polypeptides are also encompassed within the present invention. By “programmed cell death-related polypeptide” is intended a protein having the amino acid sequence set forth in SEQ ID NO: 1 (NARC10) or SEQ ID NO: 3 (NARC16), as well as fragments, biologically active portions, and variants thereof.

“Fragments” or “biologically active portions” include polypeptide fragments suitable for use as immunogens to NARC10 or NARC16 antibodies. Fragments include peptides comprising amino acid sequences sufficiently identical to or derived from the amino acid sequence of NARC10 or NARC16, or partial-length polypeptide of the invention and exhibiting at least one activity of a programmed cell death-related polypeptide, but which include fewer amino acids than the NARC10 or NARC16 full-length programmed cell death-related polypeptides disclosed herein. Typically, biologically active portions comprise a domain or motif with at least one activity of the programmed cell death-related polypeptide. A biologically active portion of a programmed cell death-related polypeptide can be a polypeptide which is, for example, 10, 25, 50, 100 or more amino acids in length. Such biologically active portions can be prepared by recombinant techniques and evaluated for one or more of the functional activities of a native programmed cell death-related polypeptide. As used herein, a fragment comprises at least 8 contiguous amino acids of SEQ ID NO: 1 or 3. The invention encompasses other fragments, however, such as any fragment in the protein greater than 8, 9, 10, 11, 12, 13, 14, or 15 amino acids.

By “variants” is intended proteins or polypeptides having an amino acid sequence that is at least about 45%, 55%, 65%, preferably about 75%, 85%, 95%, or 98% identical to the amino acid sequence of SEQ ID NO: 1 or 3. Variants also include polypeptides encoded by a nucleic acid molecule that hybridizes to the nucleic acid molecule of SEQ ID NO: 2 or 4, or a complement thereof, under stringent conditions. Such variants generally retain the functional activity of the programmed cell death-related polypeptides of the invention. Variants include polypeptides that differ in amino acid sequence due to natural allelic variation or mutagenesis.

The invention also provides programmed cell death-related chimeric or fusion proteins. As used herein, a programmed cell death-related “chimeric protein” or “fusion protein” comprises a programmed cell death-related polypeptide operably linked to a non-programmed cell death-related polypeptide. A “programmed cell death-related polypeptide” refers to a polypeptide having an amino acid sequence corresponding to a programmed cell death-related polypeptide, whereas a “non-programmed cell death-related polypeptide” refers to a polypeptide having an amino acid sequence corresponding to a protein that is not substantially identical to the programmed cell death-related polypeptide, e.g., a protein that is different from the programmed cell death-related polypeptide and which is derived from the same or a different organism. Within a programmed cell death-related fusion protein, the programmed cell death-related polypeptide can correspond to all or a portion of a programmed cell death-related polypeptide, preferably at least one biologically active portion of a programmed cell death-related polypeptide. Within the fusion protein, the term “operably linked” is intended to indicate that the programmed cell death-related polypeptide and the non-programmed cell death-related polypeptide are fused in-frame to each other. The non-programmed cell death protein-like polypeptide can be fused to the N-terminus or C-terminus of the programmed cell death protein-like polypeptide.

One useful fusion protein is a GST-programmed cell death-related fusion protein in which the programmed cell death-related sequences are fused to the C-terminus of the GST sequences. Such fusion proteins can facilitate the purification of recombinant programmed cell death-related polypeptides.

In yet another embodiment, the fusion protein is a programmed cell death protein-like-immunoglobulin fusion protein in which all or part of a programmed cell death-related polypeptide is fused to sequences derived from a member of the immunoglobulin protein family. The programmed cell death protein-like-immunoglobulin fusion proteins of the invention can be incorporated into pharmaceutical compositions and administered to a subject to inhibit an interaction between a programmed cell death-related binding protein and a programmed cell death-related polypeptide, thereby suppressing programmed cell death protein-mediated programmed cell death and cell cycle modulatory activity in vivo The programmed cell death protein-like-immunoglobulin fusion proteins can be used to affect the bioavailability of a programmed cell death-related cognate ligand. Inhibition of the programmed cell death-related ligand/programmed cell death-related interaction may be useful therapeutically, both for treating both proliferative and programmed cell death-associated disorders. Moreover, the programmed cell death protein-like-immunoglobulin fusion proteins of the invention can be used as immunogens to produce anti-NARC10 or anti-NARC16 antibodies in a subject, to purify programmed cell death-related ligands, and in screening assays to identify molecules that inhibit the interaction of a programmed cell death-related polypeptide with a programmed cell death-related ligand.

Preferably, a programmed cell death-related chimeric or fusion protein of the invention is produced by standard recombinant DNA techniques. For example, DNA fragments coding for the different polypeptide sequences may be ligated together in-frame, or the fusion gene can be synthesized, such as with automated DNA synthesizers. Alternatively, PCR amplification of gene fragments can be carried out using anchor primers that give rise to complementary overhangs between two consecutive gene fragments, which can subsequently be annealed and reamplified to generate a chimeric gene sequence (see, e.g., Ausubel et al., eds. (1995) Current Protocols in Molecular Biology) (Greene Publishing and Wiley-Interscience, NY). Moreover, a NARC10 or NARC16 programmed cell death protein-like-encoding nucleic acid can be cloned into a commercially available expression vector such that it is linked in-frame to an existing fusion moiety.

Variants of the NARC10 and NARC16 programmed cell death-related polypeptides can function as either programmed cell death-related agonists (mimetics) or as programmed cell death-related antagonists. Variants of the programmed cell death-related polypeptide can be generated by mutagenesis, e.g., discrete point mutation or truncation of the programmed cell death-related polypeptide. An agonist of the programmed cell death-related polypeptide can retain substantially the same, or a subset, of the biological activities of the naturally occurring form of the programmed cell death-related polypeptide. An antagonist of the programmed cell death-related polypeptide can inhibit one or more of the activities of the naturally occurring form of programmed cell death-related polypeptide by, for example, competitively binding to a downstream or upstream member of a cellular signaling cascade that includes the programmed cell death-related polypeptide. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the NARC10 or NARC16 programmed cell death-related polypeptides.

Variants of a programmed cell death-related polypeptide that function as either programmed cell death-related agonists or as programmed cell death-related antagonists can be identified by screening combinatorial libraries of mutants, e.g., truncation mutants, of a programmed cell death-related polypeptide for programmed cell death-related polypeptide agonist or antagonist activity. In one embodiment, a variegated library of programmed cell death-related variants is generated by combinatorial mutagenesis at the nucleic acid level and is encoded by a variegated gene library. A variegated library of programmed cell death-related variants can be produced by, for example, enzymatically ligating a mixture of synthetic oligonucleotides into gene sequences such that a degenerate set of potential programmed cell death-related sequences is expressible as individual polypeptides, or alternatively, as a set of larger fusion proteins (e.g., for phage display) containing the set of programmed cell death-related sequences therein. There are a variety of methods that can be used to produce libraries of potential programmed cell death-related variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential programmed cell death-related sequences. Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of programmed cell death-related polypeptide coding sequence can be used to generate a variegated population of programmed cell death-related fragments for screening and subsequent selection of variants of a programmed cell death-related polypeptide. In one embodiment, a library of coding sequence fragments can be generated by treating a double-stranded PCR fragment of a programmed cell death-related coding sequence with a nuclease under conditions wherein nicking occurs only about once per molecule, denaturing the double-stranded DNA, renaturing the DNA to form double-stranded DNA which can include sense/antisense pairs from different nicked products, removing single-stranded portions from reformed duplexes by treatment with S1 nuclease, and ligating the resulting fragment library into an expression vector. By this method, one can derive an expression library that encodes N-terminal and internal fragments of various sizes of the programmed cell death protein.

Several techniques are known in the art for screening gene products of combinatorial libraries made by point mutations or truncation and for screening cDNA libraries for gene products having a selected property. Such techniques are adaptable for rapid screening of the gene libraries generated by the combinatorial mutagenesis of programmed cell death-like proteins. The most widely used techniques, which are amenable to high through-pit analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique that enhances the frequency of functional mutants in the libraries, can be used in combination with the screening assays to identify programmed cell death-related variants (Arkin and Yourvan (1992) Proc. Natl. Acad. Sci. USA 89:7811-7815; Delgrave et al. (1993) Protein Engineering 6(3):327-331).

An isolated programmed cell death-related polypeptide of the invention can be used as an immunogen to generate antibodies that bind programmed cell death-related polypeptides using standard techniques for polyclonal and monoclonal antibody preparation, The full-length programmed cell death-related polypeptide can be used or, alternatively, the invention provides antigenic peptide fragments programmed cell death-related polypeptides for use as immunogens. The antigenic peptide of a programmed cell death-related polypeptide comprises at least 8, preferably 10, 15, 20, or 30 amino acid residues of the amino acid sequence shown in SEQ ID NO: 1 or SEQ ID NO: 3 and encompasses an epitope of a programmed cell death-related polypeptide such that an antibody raised against the peptide forms a specific immune complex with the programmed cell death-related polypeptide. Preferred epitopes encompassed by the antigenic peptide are regions of a programmed cell death-related polypeptide that are located on the surface of the protein, e.g., hydrophilic regions.

Accordingly, another aspect of the invention pertains to anti-programmed cell death-related polyclonal and monoclonal antibodies that bind a programmed cell death-related polypeptide. Polyclonal anti-programmed cell death-related antibodies can be prepared by immunizing a suitable subject (e.g., rabbit, goat, mouse, or other mammal) with a programmed cell death-related immunogen. The anti-programmed cell death-related titer in the immunized subject can be monitored over time by standard techniques, such as with an enzyme linked immunosorbent assay (ELISA) using immobilized programmed cell death-related polypeptide. At an appropriate time after immunization, e.g., when the anti-programmed cell death-related antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985) in Monoclonal Antibodies and Cancer Therapy, ed. Reisfeld and Sell (Alan R. Liss, Inc., New York, N.Y.), pp. 77-96) or trioma techniques. The technology for producing hybridomas is well known (see generally Coligan et al., eds. (1994) Current Protocols in Immunology (John Wiley & Sons, Inc., New York, N.Y.); Galfre et al. (1977) Nature 266:55052; Kenneth (1980) in Monoclonal Antibodies: A New Dimension In Biological Analyses (Plenum Publishing Corp., NY; and Lerner (1981) Yale J. Biol. Med., 54:387-402).

Alternative to preparing monoclonal antibody-secreting hybridomas, a monoclonal anti-programmed cell death-related antibody can be identified and isolated by screening a recombinant combinatorial immunoglobulin library (e.g., an antibody phage display library) with a programmed cell death-related polypeptide to thereby isolate immunoglobulin library members that bind the programmed cell death-related polypeptide. Kits for generating and screening phage display libraries are commercially available (e.g., the Pharmacia Recombinant Phage Antibody System, Catalog No. 27-9400-01; and the Stratagene SurfZAP 9 Phage Display Kit, Catalog No. 240612). Additionally, examples of methods and reagents particularly amenable for use in generating and screening antibody display library can be found in, for example, U.S. Pat. No. 5,223,409; PCT Publication Nos. WO 92/18619; WO 91/17271; WO 92/20791; WO 92/15679; 93/01288; WO 92/01047; 92/09690; and 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant anti-programmed cell death-related antibodies, such as chimeric and humanized monoclonal antibodies, comprising both human and nonhuman portions, which can be made using standard recombinant DNA techniques, are within the scope of the invention. Such chimeric and humanized monoclonal antibodies can be produced by recombinant DNA techniques known in the art, for example using methods described in PCT Publication Nos. WO 86/101533 and WO 87/02671; European Patent Application Nos. 184,187, 171,496, 125,023, and 173,494; U.S. Pat. Nos. 4,816,567 and 5,225,539; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; Shaw et al. (1988) J. Natl. Cancer Inst. 80:1553-1559); Morrison (1985) Science 229:1202-1207; Oi et al. (1986) Bio/Techniques 4:214; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar (1995) Int. Rev. Immunol. 13:65-93); and U.S. Pat. Nos. 5,625,126; 5,633,425; 5,569,825; 5,661,016; and 5,545,806. In addition, companies such as Abgenix, Inc. (Fremont, Calif.), can be engaged to provide human antibodies directed against a selected antigen using technology similar to that described above.

Completely human antibodies that recognize a selected epitope can be generated using a technique referred to as “guided selection.” In this approach a selected non-human monoclonal antibody, e.g., a murine antibody, is used to guide the selection of a completely human antibody recognizing the same epitope. This technology is described by Jespers et al. (1994) Bio/Technology 12:899-903).

An anti-programmed cell death-related polypeptide antibody (e.g., monoclonal antibody) can be used to isolate programmed cell death-related polypeptides by standard techniques, such as affinity chromatography or immunoprecipitation. An anti-programmed cell death-related polypeptide antibody can facilitate the purification of natural programmed cell death-related polypeptides from cells and of recombinantly produced programmed cell death-related polypeptides expressed in host cells. Moreover, an anti-programmed cell death-related polypeptide antibody can facilitate the purification of natural programmed cell death-related polypeptides from cells and of recombinantly produced programmed cell death-related polypeptide expressed in host cells. Anti-programmed cell death-related polypeptide antibodies can be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, or ³H.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrorie, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and loniustine (CCNU), cyclothosphamide, busulfan, dibromomannitol, streptozolocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84:Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

III. Recombinant Expression Vectors and Host Cells

Another aspect of the invention pertains to vectors, preferably expression vectors, containing a nucleic acid encoding a NARC10 or NARC16 programmed cell death-related polypeptide (or a portion thereof). “Vector” refers to a nucleic acid molecule capable of transporting another nucleic acid to which it has been linked, such as a “plasmid”, a circular double-stranded DNA loop into which additional DNA segments can be ligated, or a viral vector, where additional DNA segments can be ligated into the viral genome. The vectors are useful for autonomous replication in a host cell or may be integrated into the genome of a host cell upon introduction into the host cell, and thereby are replicated along with the host genome (e.g., nonepisomal mammalian vectors). Expression vectors are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses, and adeno-associated viruses), that serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, operably linked to the nucleic acid sequence to be expressed. “Operably linked” is intended to mean that the nucleotide-sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to include promoters, enhancers, and other expression control elements (e.g., polyadenylation signals). See, for example, Goeddel (1990) in Gene Expression Technology: Methods in Enzymology 185 (Academic Press, San Diego, Calif.). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., programmed cell death-related polypeptides, mutant forms of programmed cell death-related polypeptides, fusion proteins, etc.).

The recombinant expression vectors of the invention can be designed for expression of programmed cell death-related polypeptide in prokaryotic or eukaryotic host cells. Expression of proteins in prokaryotes is most often carried out in E. coli with vectors containing constitutive or inducible promoters directing the expression of either fusion or nonfusion proteins. Fusion vectors add a number of amino acids to a protein encoded therein, usually to the amino terminus of the recombinant protein. Typical fusion expression vectors include pGEX (Pharmacia Biotech Inc; Smith and Johnson (1988) Gene 67:31-40), pMAL (New England Biolabs, Beverly, Mass.), and pRIT5 (Pharmacia, Piscataway, N.J.) which fuse glutathione S-transferase (GST), maltose E binding protein, or protein A, respectively, to the target recombinant protein. Examples of suitable inducible nonfusion E. coli expression vectors include pTrc (Amann et al. (1988) Gene 69:301-315) and pET 11d (Studier et al. (1990) in Gene Expression Technology: Methods in Enzymology 185 (Academic Press, San Diego, Calif.), pp. 60-89). Strategies to maximize recombinant protein expression in E. coli can be found in Gottesman (1990) in Gene Expression Technology: Methods in Enzymology 185 (Academic Press, CA), pp. 119-128 and Wada et al. (1992) Nucleic Acids Res. 20:2111-2118. Target gene expression from the pTrc vector relies on host RNA polymerase transcription from a hybrid trp-lac fusion promoter.

Suitable eukaryotic host cells include insect cells (examples of Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39)); yeast cells (examples of vectors for expression in yeast S. cereivisiae include pYepSec1 (Baldari et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif.), and pPicZ (Invitrogen Corporation, San Diego, Calif.)); or mammalian cells (mammalian expression vectors include pCDM8 (Seed (1987) Nature 329:840) and pMT2PC (Kaufman et al. (1987) EMBO J. 6:187:195)). Suitable mammalian cells include Chinese hamster ovary cells (CHO) or COS cells. In mammalian cells, the expression vector's control functions are often provided by viral regulatory elements. For example, commonly used promoters are derived from polyoma, Adenovirus 2, cytomegalovirus, and Simian Virus 40. For other suitable expression systems for both prokaryotic and eukaryotic cells, see chapters 16 and 17 of Sambrook et al. (1989) Molecular cloning: A Laboratory Manual (2d ed., Cold Spring Harbor Laboratory Press, Plainview, N.Y.). See, Goeddel (1990) in Gene Expression Technology: Methods in Enzymology 185 (Academic Press, San Diego, Calif.). Alternatively, the recombinant expression vector can be transcribed and translated in vitro, for example using T7 promoter regulatory sequences and T7 polymerase.

The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell but are still included within the scope of the term as used herein.

In one embodiment, the expression vector is a recombinant mammalian expression vector that comprises tissue-specific regulatory elements that direct expression of the nucleic acid preferentially in a particular cell type. Suitable tissue-specific promoters include the albumin promoter (e.g., liver-specific promoter; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Patent Publication No. 264,166). Developmentally-regulated promoters are also encompassed, for example the murine hox homeobox promoters (Kessel and Gruss (1990) Science 249:374-379), the α-fetoprotein promoter (Campes and Tilghman (1989) Genes Dev. 3:537-546), and the like.

The invention further provides a recombinant expression vector comprising a DNA molecule of the invention cloned into the expression vector in an antisense orientation. That is, the DNA molecule is operably linked to a regulatory sequence in a manner that allows for expression (by transcription of the DNA molecule) of an RNA molecule that is antisense to programmed cell death-related mRNA. Regulatory sequences operably linked to a nucleic acid cloned in the antisense orientation can be chosen to direct the continuous expression of the antisense RNA molecule in a variety of cell types, for instance viral promoters and/or enhancers, or regulatory sequences can be chosen to direct constitutive, tissue-specific, or cell-type-specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (1986) Reviews—Trends in Genetics, Vol. 1(1).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or e lectroporation. Suitable methods for transforming or transfecting host cells can be found in Sambrook et al. (1989) Molecular Cloning: A Laboratory Manual (2d ed., Cold Spring Harbor Laboratory Press, Plainview, N.Y.) and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin, and methotrexate. Nucleic acid encoding a selectable marker can be introduced into a host cell on the same vector as that encoding a programmed cell death-like protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) programmed cell death-related polypeptide. Accordingly, the invention further provides methods for producing programmed cell death-related polypeptide using the host cells of the invention. In one embodiment, the method comprises culturing the host cell of the invention, into which a recombinant expression vector encoding a programmed cell death-related polypeptide has been introduced, in a suitable medium such that programmed cell death-related polypeptide is produced. In another embodiment, the method further comprises isolating programmed cell death-related polypeptide from the medium or the host cell.

The host cells of the invention can also be used to produce nonhuman transgenic animals. For example, in one embodiment, a host cell of the invention is a fertilized oocyte or an embryonic stem cell into which programmed cell death protein-like-coding sequences have been introduced. Such host cells can then be used to create nonhuman transgenic animals in which exogenous programmed cell death-related sequences have been introduced into their genome or homologous recombinant animals in which endogenous programmed cell death-related sequences have been altered. Such animals are useful for studying the function and/or activity of programmed cell death-related genes and proteins and for identifying and/or evaluating modulators of programmed cell death-related activity. As used herein, a “transgenic animal” is a nonhuman animal, preferably a mammal, more preferably a rodent such as a rat or mouse, in which one or more of the cells of the animal includes a transgene. Other examples of transgenic animals include nonhuman primates, sheep, dogs, cows, goats, chickens, amphibians, etc. A transgene is exogenous DNA that is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a nonhuman animal, preferably a mammal, more preferably a mouse, in which an endogenous programmed cell death-related gene has been altered by homologous recombination between the endogenous gene and an exogenous DNA molecule introduced into a cell of the animal, e.g., an embryonic cell of the animal, prior to development of the animal.

A transgenic animal of the invention can be created by introducing programmed cell death protein-like-encoding nucleic acid into the male pronuclei of a fertilized oocyte, e.g., by microinjection, retroviral infection, and allowing the oocyte to develop in a pseudopregnant female foster animal. The programmed cell death-related cDNA sequence can be introduced as a transgene into the genome of a nonhuman animal. Alternatively, a homologue of the mouse programmed cell death-related gene can be isolated based on hybridization and used as a transgene. Intronic sequences and polyadenylation signals can also be included in the transgene to increase the efficiency of expression of the transgene. A tissue-specific regulatory sequence(s) can be operably linked to the programmed cell death-related transgene to direct expression of programmed cell death-related polypeptide to particular cells. Methods for generating transgenic animals via embryo manipulation and microinjection, particularly animals such as mice, have become conventional in the art and are described, for example, in U.S. Pat. Nos. 4,736,866, 4,870,009, and 4,873,191 and in Hogan (1986) Manipulating the Mouse Embryo (Cold Spring Harbor Laboratory Press, Cold Spring Harbor, N.Y., 1986). Similar methods are used for production of other transgenic animals. A transgenic founder animal can be identified based upon the presence of the programmed cell death-related transgene in its genome and/or expression programmed cell death-related mRNA in tissues or cells of the animals. A transgenic founder animal can then be used to breed additional animals carrying the transgene. Moreover, transgenic animals carrying a transgene encoding programmed cell death-related gene can further be bred to other transgenic animals carrying other transgenes.

To create a homologous recombinant animal, one prepares a vector containing at least a portion of programmed cell death-related gene or a homolog of the gene into which a deletion, addition, or substitution has been introduced to thereby alter, e.g., functionally disrupt, the programmed cell death-related gene. In a preferred embodiment, the vector is designed such that, upon homologous recombination, the endogenous programmed cell death-related gene is functionally disrupted (i.e., no longer encodes a functional protein; also referred to as a “knock out” vector). Alternatively, the vector can be designed such that, upon homologous recombination, the endogenous programmed cell death-related gene is mutated or otherwise altered but still encodes functional protein (e.g., the upstream regulatory region can be altered to thereby alter the expression of the programmed cell death-related polypeptide). In the homologous recombination vector, the altered portion of programmed cell death-related gene is flanked at its 5N and 3N ends by additional nucleic acid of the programmed cell death-related gene to allow for homologous recombination to occur between the exogenous programmed cell death-related gene carried by the vector and an endogenous programmed cell death-related gene in an embryonic stem cell. The additional flanking programmed cell death-related nucleic acid is of sufficient length for successful homologous recombination with the endogenous gene. Typically, several kilobases of flanking DNA (at both the 5′ and 3′ ends) are included in the vector (see, e.g., Thomas and Capecchi (1987) Cell 51:503 for a description of homologous recombination vectors). The vector is introduced into an embryonic stem cell line (e.g., by electroporation), and cells in which the introduced programmed cell death-related gene has homologously recombined with the endogenous programmed cell death-related gene are selected (see, e.g., Li et al. (1992) Cell 69:915). The selected cells are then injected into a blastocyst of an animal (e.g., a mouse) to form aggregation chimeras (see, e g., Bradley (1987) in Teratocarcinomas and Embryonic Stem Cells: A Practical Approach, ed. Robertson (IRL, Oxford pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication Nos. WO 90/11354, WO 91/01140, WO 92/0968, and WO 93/04169.

In another embodiment, transgenic nonhuman animals containing selected systems that allow for regulated expression of the transgene can be produced. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the nonhuman transgenic animals described herein can also be produced according to the methods described in Wilmut et al. (1997) Nature 385:810-813 and PCT Publication Nos. WO 97/07668 and WO 97/07669.

IV. Pharmaceutical Compositions

The programmed cell death-related nucleic acid molecules, programmed cell death-related polypeptides, and anti-programmed cell death-related antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The compositions of the invention are useful to treat any of the disorders discussed herein. Treatment is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes and antisense oligonucleotides.

The pharmaceutical compositions are provided in therapeutically effective amounts. By “therapeutically effective amounts” is intended an amount sufficient to modulate the desired response. As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypertide, or antibody can include a single treatment or, preferably, can include a series of treatments. In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents depends upon a number of factors within the knowledge of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetramino acidcetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF; Parsippany, N.J.), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example., by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride, in the composition. Prolonged absorption of the injectable compositions can be brought about by including in the composition an agent that delays absorption, for example, aluminum monostearate and gelatin.

Sterile injectable solutions can be prepared by incorporating the active compound (e.g., a programmed cell death-like protein or anti-programmed cell death-like antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying, which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth, or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compounds are delivered in the form of an aerosol spray from a pressurized container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated with each unit containing a predetermined quantity of active compound calculated to produce the desired therapeutic effect in association with the required pharmaceutical carrier. Depending on the type and severity of the disease, about 1 μg/kg to about 15 mg/kg (e.g., 0.1 to 20 mg/kg) of antibody is an initial candidate dosage for administration to the patient, whether, for example, by one or more separate administrations, or by continuous infusion. A typical daily dosage might range from about 1 μg/kg to about 100 mg/kg or more, depending on the factors mentioned above. For repeated administrations, over several days or longer, depending on the condition, the treatment is sustained until a desired suppression of disease symptoms occurs. However, other dosage regimens may be useful. The progress of this therapy is easily monitored by conventional techniques and assays. An exemplary dosing regimen is disclosed in WO 94/04188. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, and antibodies described herein can be used in one or more of the following methods: (a) screening assays; (b) detection assays (e.g., chromosomal mapping, tissue typing, forensic biology); (c) predictive medicine (e.g., diagnostic assays, prognostic assays, monitoring clinical trials, and pharmacogenomics); and (d) methods of treatment (e.g., therapeutic and prophylactic). The isolated nucleic acid molecules of the invention can be used to express programmed cell death-related polypeptide (e.g., via a recombinant expression vector in a host cell in gene therapy applications), to detect programmed cell death-related mRNA (e.g., in a biological sample) or a genetic lesion in a programmed cell death-related gene, and to modulate programmed cell death-related activity. In addition, the programmed cell death-related polypeptides can be used to screen drugs or compounds that modulate programmed cell death as well as to treat disorders characterized by insufficient or excessive production of programmed cell death-related polypeptide or production of programmed cell death-related polypeptide forms that have decreased or aberrant activity compared to programmed cell death-related wild type protein. In addition, the anti-programmed cell death-related polypeptide antibodies of the invention can be used to detect and isolate programmed cell death-related polypeptides and modulate programmed cell death-related activity.

A. Screening Assays

The invention provides a method (also referred to herein as a “screening assay”) for identifying modulators, i.e., candidate or test compounds or agents (e.g., peptides, peptidomimetics, small molecules, or other drugs) that bind programmed cell death-related polypeptides or have a stimulatory or inhibitory effect on, for example, programmed cell death-related expression or programmed cell death-related activity.

The test compounds of the present invention can be obtained using any of the numerous approaches in combinatorial library methods known in the art, including biological libraries, spatially addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, nonpeptide oligomer, or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckennann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869), or phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310).

Determining the ability of the test compound to bind to the programmed cell death-related polypeptide can be accomplished, for example, by coupling the test compound with a radioisotope or enzymatic label such that binding of the test compound to the programmed cell death-related polypeptide or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

In a similar manner, one may determine the ability of the programmed cell death-related polypeptide to bind to or interact with a programmed cell death-related target molecule. By “target molecule” is intended a molecule with which programmed cell death-related polypeptide binds or interacts in nature. In a preferred embodiment, the ability of the programmed cell death-related polypeptide to bind to or interact with a programmed cell death-related target molecule can be determined by monitoring the activity of the target molecule. For example, the activity of the target molecule can be monitored by assaying for the number of cells undergoing programmed cell death, catalytic/enzymatic activity of the target on an appropriate substrate, detecting the induction of a reporter gene (e.g., a programmed cell death protein-like-responsive regulatory element operably linked to a nucleic acid encoding a detectable marker, e.g. luciferase), or detecting a cellular response, for example, cellular differentiation or cell proliferation.

In yet another embodiment, an assay of the present invention is a cell-free assay comprising contacting a programmed cell death-related polypeptide or biologically active portion thereof with a test compound and determining the ability of the test compound to bind to the programmed cell death-related polypeptide or biologically active portion thereof. Binding of the test compound to the programmed cell death-related polypeptide can be determined either directly or indirectly as described above. In a preferred embodiment, the assay includes contacting the programmed cell death-related polypeptide or biologically active portion thereof with a known compound that binds programmed cell death-related polypeptide to form an assay mixture, contacting the assay mixture with a test compound, and determining the ability of the test compound to preferentially bind to programmed cell death-related polypeptide or biologically active portion thereof as compared to the known compound.

In another embodiment, an assay is a cell-free assay that comprises the steps of contacting programmed cell death-related polypeptide or biologically active portion thereof with a test compound and determining the ability of the test compound to modulate (e.g., stimulate or inhibit) the activity of the programmed cell death-related polypeptide or biologically active portion thereof. Determining the ability of the test compound to modulate the activity of a programmed cell death-related polypeptide can be accomplished, for example, by determining the ability of the programmed cell death-related polypeptide to bind to a programmed cell death-related target molecule as described above for determining direct binding. In an alternative embodiment, determining the ability of the test compound to modulate the activity of a programmed cell death-related polypeptide can be accomplished by determining the ability of the programmed cell death-related polypeptide to further modulate a programmed cell death-related target molecule. For example, the catalytic/enzymatic activity of the target molecule on an appropriate substrate can be determined as previously described.

In yet another embodiment, the cell-free assay comprises the steps of contacting the programmed cell death-related polypeptide or biologically active portion thereof with a known compound that builds a programmed cell death-related polypeptide to form an assay mixture, contacting the assay mixture with a test compound, and determining the ability of the test compound to preferentially bind to or modulate the activity of a programmed cell death-related target molecule.

In the above-mentioned assays, it may be desirable to immobilize either a programmed cell death-related polypeptide or its target molecule to facilitate separation of complexed from uncomplexed forms of one or both of the proteins, as well as to accommodate automation of the assay. In one embodiment, a fusion protein can be provided that adds a domain that allows one or both of the proteins to be bound to a matrix. For example, glutathione-S-transferase/programmed cell death-like fusion proteins or glutathione-S-transferase/target fusion proteins can be adsorbed onto glutathione sepharose beads (Sigma Chemical, St. Louis, Mo.) or glutathione-derivatized microtitre plates, which are then combined with the test compound or the test compound and either the nonadsorbed target protein or programmed cell death-related polypeptide, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of programmed cell death-related binding or activity determined using standard techniques.

Other techniques for immobilizing proteins on matrices can also be used in the screening assays of the invention. For example, either programmed cell death-related polypeptide or one of their target molecules can be immobilized utilizing conjugation of biotin and streptavidin. Biotinylated programmed cell death-related molecules or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96-well plates (Pierce Chemicals). Alternatively, antibodies reactive with a programmed cell death-related polypeptide or target molecules but which do not interfere with binding of the programmed cell death-related polypeptide to its target molecule can be derivatized to the wells of the plate, and unbound target or programmed cell death-related polypeptide trapped in the wells by antibody conjugation. Methods for detecting such complexes, in addition to those described above for the GST-immobilized complexes, include immunodetection of complexes using antibodies reactive with programmed cell death-related polypeptide or target molecule, as well as enzyme-linked assays that rely on detecting an enzymatic activity associated with the programmed cell death-related polypeptide or target molecule.

In another embodiment, modulators of programmed cell death-related expression are identified in a method in which a cell is contacted with a candidate compound and the expression of programmed cell death-related mRNA or protein in the cell is determined relative to expression of programmed cell death-related mRNA or protein in a cell in the absence of the candidate compound. When expression is greater (statistically significantly greater) in the presence of the candidate compound than in its absence, the candidate compound is identified as a stimulator of programmed cell death-related mRNA or protein expression. Alternatively, when expression is less (statistically significantly less) in the presence of the candidate compound than in its absence, the candidate compound is identified as an inhibitor of programmed cell death-related mRNA or protein expression. The level programmed cell death-related mRNA or protein expression in the cells can be determined by methods described herein for detecting programmed cell death-related mRNA or protein.

In yet another aspect of the invention, programmed cell death-related polypeptides can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300), to identify other proteins, which bind to or interact with programmed cell death-related polypeptide (“programmed cell death-related binding proteins” or “programmed cell death-related bp”) and modulate programmed cell death-related activity. Such programmed cell death protein-like-binding proteins are also likely to be involved in the propagation of signals by the programmed cell death-related polypeptides as, for example, upstream or downstream elements of programmed cell death-related pathway.

This invention further pertains to novel agents identified by the above-described screening assays and uses thereof for treatments as described herein.

B. Detection Assays

Portions or fragments of the cDNA sequences identified herein (and the corresponding complete gene sequences) can be used in numerous ways as polynucleotide reagents. For example, these sequences can be used to: (1) map their respective genes on a chromosome; (2) identify an individual from a minute biological sample (tissue typing); and (3) aid in forensic identification of a biological sample. These applications are described in the subsections below.

1. Chromosome Mapping

The isolated complete or partial programmed cell death-like gene sequences of the invention can be used to map their respective programmed cell death-related genes on a chromosome, thereby facilitating the location of gene regions associated with genetic disease. Computer analysis of programmed cell death-related sequences can be used to rapidly select PCR primers (preferably 15-25 bp in length) that do not span more than one exon in the genomic DNA, thereby simplifying the amplification process. These primers can then be used for PCR screening of somatic cell hybrids containing individual human chromosomes. Only those hybrids containing the human gene corresponding to the programmed cell death-related sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow (because they lack a particular enzyme), but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes (D'Eustachio et al. (1983) Science 220:919-924). Somatic cell hybrids containing only fragments of human chromosomes can also be produced by using human chromosomes with translocations and deletions.

Other mapping strategies that can similarly be used to map programmed cell death-related sequence to its chromosome include in situ hybridization (described in Fan et al. (1990) Proc. Natl. Acad. Sci. USA 87:6223-27), pre-screening with labeled flow-sorted chromosomes, and pre-selection by hybridization to chromosome specific cDNA libraries. Furthermore, fluorescence in situ hybridization (FISH) of a DNA sequence to a metaphase chromosomal spread can be used to provide a precise chromosomal location in one step. For a review of this technique, see Verma eta a. (1988) Human Chromosomes: A Manual of Basic Techniques (Pergamon Press, NY). The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results in a reasonable amount of time.

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Another strategy to map the chromosomal location programmed cell death-related genes uses programmed cell death-related polypeptides and fragments and sequences of the present invention and antibodies specific thereto. This mapping can be carried out by specifically detecting the presence of a programmed cell death-related polypeptide in members of a panel of somatic cell hybrids between cells of a first species of animal from which the protein originates and cells from a second species of animal, and then determining which somatic cell hybrid(s) expresses the polypeptide and noting the chromosomes(s) from the first species of animal that it contains. For examples of this technique, see Pajunen et al. (1988) Cytogenet. Cell. Genet. 47:37-41 and Van Keuren et al. (1986) Hum. Genet. 74:34-40. Alternatively, the presence of a programmed cell death-related polypeptide in the somatic cell hybrids can be determined by assaying an activity or property of the polypeptide, for example, enzymatic activity, as described in Bordelon-Riser et al. (1979) Somatic Cell Genetics 5:597-613 and Owerbach et al. (1978) Proc. Natl. Acad. Sci. USA 75:5640-5644.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland et al. (1987) Nature 325:783-787.

Moreover, differences in the DNA sequences between individuals affected and unaffected with a disease associated with programmed cell death-related gene can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The programmed cell death-related sequences of the present invention can also be used to identify individuals from minute biological samples. The United States military, for example, is considering, the use of restriction fragment length polymorphism (RFLP) for identification of its personnel. In this technique, an individual's genomic DNA is digested with one or more restriction enzymes and probed on a Southern blot to yield unique bands for identification. The sequences of the present invention are useful as additional DNA markers for RFLP (described, e.g., in U.S. Pat. No. 5,272,057).

Furthermore, the sequences of the present invention can be used to provide an alternative technique for determining the actual base-by-base DNA sequence of selected portions of an individual's genome. Thus, the programmed cell death-related sequences of the invention can be used to prepare two PCR primers from the 5N and 3N ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The programmed cell death-related sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. The noncoding sequences of SEQ ID NO: 2 or 4 can comfortably provide positive individual identification with a panel of perhaps 10 to 1,000 primers that each yield a noncoding amplified sequence of 100 bases. If a predicted coding sequence, such as that in SEQ ID NO: 2 or 4, is used, a more appropriate number of primers for positive individual identification would be 500 to 2,000.

3. Use of Partial Programmed Cell Death-Related Sequences in Forensic Biology

DNA-based identification techniques can also be used in forensic biology. In this manner, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” that is unique to a particular individual. As mentioned above, actual base sequence information can be used for identification as an accurate alternative to patterns formed by restriction enzyme generated fragments. Sequences targeted to noncoding regions of SEQ ID NO: 2 or SEQ ID NO: 4 are particularly appropriate for this use as greater numbers of polymorphisms occur in the noncoding regions, making it easier to differentiate individuals using this technique. Examples of polynucleotide reagents include the programmed cell death-related sequences or portions thereof, e.g., fragments derived from the noncoding regions of SEQ ID NO: 2 or SEQ ID NO: 4 having a length of at [east 20 or 30 bases.

The programmed cell death-related sequences described herein can further be used to provide polynucleotide reagents, e.g., labeled or labelable probes that can be used in, for example, an in situ hybridization technique, to identify a specific tissue. This can be very useful in cases where a forensic pathologist is presented with a tissue of unknown origin. Panels of such programmed cell death-related probes, can be used to identify tissue by species and/or by organ type.

In a similar fashion, these reagents, e.g., programmed cell death-related primers or probes can be used to screen tissue culture for contamination (i.e., screen for the presence of a mixture of different types of cells in a culture).

C. Predictive Medicine

The present invention also pertains to the field of predictive medicine in which diagnostic assays, prognostic assays, pharmacogenomics, and monitoring clinical trails are used for prognostic (predictive) purposes to thereby treat an individual prophylactically. These applications are described in the subsections below.

1. Diagnostic Assays

One aspect of the present invention relates to diagnostic assays for detecting programmed cell death-related polypeptide and/or nucleic acid expression as well as programmed cell death-related activity, in the context of a biological sample. An exemplary method for detecting the presence or absence of programmed cell death-related polypeptides in a biological sample involves obtaining a biological sample from a test subject and contacting the biological sample with a compound or an agent capable of detecting programmed cell death-related polypeptide or nucleic acid (e.g., mRNA, genomic DNA) that encodes programmed cell death-related polypeptide such that the presence of programmed cell death-related polypeptide is detected in the biological sample. Results obtained with a biological sample from the test subject may be compared to results obtained with a biological sample from a control subject.

A preferred agent for detecting programmed cell death-related mRNA or genomic DNA is a labeled nucleic acid probe capable of hybridizing programmed cell death-related mRNA or genomic DNA. The nucleic acid probe can be, for example, a full-length programmed cell death-related nucleic acid, such as the nucleic acids of SEQ ID NO: 2, SEQ ID NO: 4, or a portion thereof, such as a nucleic acid molecule of at least 15, 30, 50, 100, 250, or 500 nucleotides in length and sufficient to specifically hybridize under stringent conditions to NARC10 or NARC16 mRNA or genomic DNA. Other suitable probes for use in the diagnostic assays of the invention are described herein.

A preferred agent for detecting programmed cell death-related polypeptide is an antibody capable of binding to programmed cell death-related polypeptide, preferably an antibody with a detectable label. Antibodies can be polyclonal, or more preferably, monoclonal. An intact antibody, or a fragment thereof (e.g., Fab or F(abN)₂ )can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The term “biological sample” is intended to include tissues, cells, and biological fluids isolated from a subject, as well as tissues, cells, and fluids present within a subject. That is, the detection method of the invention can be used to detect programmed cell death-related mRNA, protein, or genomic DNA in a biological sample in vitro as well as in vivo. For example, in vitro techniques for detection of programmed cell death-related mRNA include Northern hybridizations and in situ hybridizations. In vitro techniques for detection of programmed cell death-related polypeptide include enzyme linked immunosorbent assays (ELISAs), Western blots, immunoprecipitations, and immunofluorescence. In vitro techniques for detection of programmed cell death-related genomic DNA include Southern hybridizations. Furthermore, in vivo techniques for detection of programmed cell death-related polypeptide include introducing into a subject a labeled anti-programmed cell death-related antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject.

The invention also encompasses kits for detecting the presence of programmed cell death-related polypeptides in a biological sample (a test sample). Such kits can be used to determine if a subject is suffering from or is at increased risk of developing a disorder associated with aberrant expression of programmed cell death-related polypeptide (e.g., an neurodegenerative disorder). For example, the kit can comprise a labeled compound or agent capable of detecting programmed cell death-related polypeptide or mRNA in a biological sample and means for determining the amount of a programmed cell death protein-like protein in the sample (e.g., an anti-programmed cell death-related antibody or an oligonucleotide probe that binds to DNA encoding a programmed cell death-related polypeptide, e.g., SEQ ID NO: 1 and SEQ ID NO: 3). Kits can also include instructions for observing that the tested subject is suffering from or is at risk of developing a disorder associated with aberrant expression of programmed cell death-related sequences if the amount of programmed cell death-related polypeptide or mRNA is above or below a normal level.

For antibody-based kits, the kit can comprise, for example: (1) a first antibody (e.g., attached to a solid support) that binds to programmed cell death-related polypeptide; and, optionally, (2) a second, different antibody that binds to programmed cell death-related polypeptide or the first antibody and is conjugated to a detectable agent. For oligonucleotide-based kits, the kit can comprise, for example: (1) an oligonucleotide, e.g., a detectably labeled oligonucleotide, that hybridizes to a programmed cell death-related nucleic acid sequence or (2) a pair of primers useful for amplifying a programmed cell death-related nucleic acid molecule.

The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples that can be assayed and compared to the test sample contained. Each component of the kit is usually enclosed within an individual container, and all of the various containers are within a single package along with instructions for observing whether the tested subject is suffering from or is at risk of developing a disorder associated with aberrant expression of programmed cell death-related polypeptides.

2. Other Diagnostic Assays

In another aspect, the invention features a method of analyzing a plurality of capture probes. The method can be used, e.g., to analyze gene expression. The method includes: providing a two Dimensional array having a plurality of addresses, each address of the plurality being positionally distinguishable from each other address of the plurality, and each address of the plurality having a unique capture probe, e.g., a nucleic acid or peptide sequence; contacting the array with programmed cell death-related nucleic acid, preferably purified, polypeptide, preferably purified, or antibody, and thereby evaluating the plurality of capture probes. Binding, e.g., in the case of a nucleic acid, hybridization, with a capture probe at an address of the plurality, is detected, e.g., by signal generated from a label attached to the programmed cell death-related nucleic acid, polypeptide, or antibody. The capture probes can be a set of nucleic acids from a selected sample, e.g., a sample of nucleic acids derived from a control or non-stimulated tissue or cell.

The method can include contacting programmed cell death-related nucleic acid, polypeptide, or antibody with a first array having a plurality of capture probes and a second array having a different plurality of capture probes. The results of each hybridization can be compared, e.g., to analyze differences in expression between a first and second sample. The first plurality of capture probes can be from a control sample, e.g., a wild type, normal, or non-diseased, non-stimulated, sample, e.g., a biological fluid, tissue, or cell sample. The second plurality of capture probes can be from an experimental sample, e.g., a mutant type, at risk, disease-state or disorder-state, or stimulated, sample, e.g., a biological fluid, tissue, or cell sample.

The plurality of capture probes can be a plurality of nucleic acid probes each of which specifically hybridizes, with an allele of a programmed cell death-related sequence of the invention. Such methods can be used to diagnose a subject, e.g., to evaluate risk for a disease or disorder, to evaluate suitability of a selected treatment for a subject, to evaluate whether a subject has a disease or disorder. Thus, for example, the NARC10 sequence set forth in SEQ ID NO: 2 encodes a nucleosome assembly protein-like polypeptide that is associated with chromatin assembly, cell cycle, and programmed cell death and thus is useful for evaluating cell proliferation and apoptotic disorders. The NARC16 sequence set forth in SEQ ID NO: 4 encodes a glycerophosphodiester phosphodiesterase-like polypeptide and is involved in glycerol phosphoryl diester hydrolysis, cell cycle control, and programmed cell death and thus is useful for evaluating cell proliferation and apoptotic disorders.

The method can be used to detect single nucleotide polymorphisms (SNPs), as described below.

In another aspect, the invention features a method of analyzing a plurality of probes. The method is useful, e.g., for analyzing gene expression. The method includes: providing a two dimensional array having a plurality of addresses, each address of the plurality being positionally distinguishable from each other address of the plurality having a unique capture probe, e.g., wherein the capture probes are from a cell or subject which express a programmed cell death-related polypeptide of the invention or from a cell or subject in which a programmed cell death protein-like-mediated response has been elicited, e.g., by contact of the cell with a programmed cell death-related nucleic acid or protein of the invention, or administration to the cell or subject a programmed cell death-related nucleic acid or protein of the invention; contacting the array with one or more inquiry probes, wherein an inquiry probe can be a nucleic acid, polypeptide, or antibody (which is preferably other than a programmed cell death-related nucleic acid, polypeptide, or antibody of the invention); providing a two dimensional array having a plurality of addresses, each address of the plurality being positionally distinguishable from each other address of the plurality, and each address of the plurality having a unique capture probe, e.g., wherein the capture probes are from a cell or subject which does not express a programmed cell death-related sequence of the invention (or does not express as highly as in the case of the programmed cell death-related positive plurality of capture probes) or from a cell or subject in which a programmed cell death protein-like-mediated response has not been elicited (or has been elicited to a lesser extent than in the first sample); contacting the array with one or more inquiry probes (which is preferably other than a programmed cell death-related nucleic acid, polypeptide, or antibody of the invention), and thereby evaluating the plurality of capture probes. Binding, e.g., in the case of a nucleic acid, hybridization, with a capture probe at an address of the plurality, is detected, e.g., by signal generated from a label attached to the nucleic acid, polypeptide, or antibody.

In another aspect, the invention features a method of analyzing a programmed cell death-related sequence of the invention, e.g., analyzing structure, function, or relatedness to other nucleic acid or amino acid sequences. The method includes: providing a programmed cell death-related nucleic acid or amino acid sequence, e.g., the NARC10 or NARC16 sequence set forth in SEQ ID NO: 1 and SEQ ID NO: 3, respectively, or a portion thereof; comparing the programmed cell death-related sequence with one or more preferably a plurality of sequences from a collection of sequences, e.g., a nucleic acid or protein sequence database; to thereby analyze the programmed cell death-related sequence of the invention.

The method can include evaluating the sequence identity between a programmed cell death-related sequence of the invention, e.g., the NARC10 or NARC16 sequence, and a database sequence. The method can be performed by accessing the database at a second site, e.g., over the internet.

In another aspect, the invention features, a set of oligonucleotides, useful, e.g., for identifying SNP's, or identifying specific alleles of a programmed cell death-related sequence of the invention, e.g., the NARC10 or NARC16 sequence. The set includes a plurality of oligonucleotides, each of which has a different nucleotide at an interrogation position, e.g., an SNP or the site of a mutation. In a preferred embodiment, the oligonucleotides of the plurality identical in sequence with one another (except for differences in length). The oligonucleotides can be provided with differential labels, such that an oligonucleotides which hybridizes to one allele provides a signal that is distinguishable from an oligonucleotides which hybridizes to a second allele.

3. Prognostic Assays

The methods described herein can furthermore be utilized as diagnostic or prognostic assays to identify subjects having or at risk of developing a disease or disorder associated with NARC10 or NARC16 programmed cell death-related polypeptides, NARC10 or NARC16 programmed cell death-related nucleic acid expression, or NARC10 or NARC16 programmed cell death-related activity. Prognostic assays can be used for prognostic or predictive purposes to thereby prophylactically treat an individual prior to the onset of a disorder characterized by or associated with programmed cell death-related polypeptide, programmed cell death-related nucleic acid expression, or programmed cell death-related activity.

Thus, the present invention provides a method in which a test sample is obtained from a subject, and programmed cell death-related polypeptide or nucleic acid (e.g., mRNA, genomic DNA) is detected, wherein the presence of programmed cell death-related polypeptide or nucleic acid is diagnostic for a subject having or at risk of developing a disease or disorder associated with aberrant programmed cell death-related expression or activity. As used herein, a “test sample” refers to a biological sample obtained from a subject of interest. For example, a test sample can be a biological fluid (e.g., serum), cell sample, or tissue.

Furthermore, using the prognostic assays described herein, the present invention provides methods for determining whether a subject can be administered a specific agent (e.g., an agonist, antagonist, peptidomimetic, protein, peptide, nucleic acid, small molecule, or other drug candidate) or class of agents (e.g., agents of a type that decrease programmed cell death-related activity) to effectively treat a disease or disorder associated with aberrant programmed cell death-related expression or activity. In this manner, a test sample is obtained and programmed cell death-related polypeptide or nucleic acid is detected. The presence of programmed cell death-related polypeptide or nucleic acid is diagnostic for a subject that can be administered the agent to treat a disorder associated with aberrant programmed cell death-related expression or activity.

The methods of the invention can also be used to detect genetic lesions or mutations in a programmed cell death-related gene, thereby determining if a subject with the lesioned gene is at risk for a disorder characterized by aberrant programmed cell death. In preferred embodiments, the methods include detecting, in a sample of cells from the subject, the presence or absence of a genetic lesion or mutation characterized by at least one of an alteration affecting the integrity of a gene encoding a programmed cell death protein-like-protein, or the misexpression of the programmed cell death-related gene. For example, such genetic lesions or mutations can be detected by ascertaining the existence of at least one of: (1) a deletion of one or more nucleotides from a programmed cell death-related gene; (2) an addition of one or more nucleotides to a programmed cell death-related gene; (3) a substitution of one or more nucleotides of a programmed cell death-related gene; (4) a chromosomal rearrangement of a programmed cell death-related gene; (5) an alteration in the level of a messenger RNA transcript of a programmed cell death-related gene; (6) an aberrant modification of a programmed cell death-related gene, such as of the methylation pattern of the genomic DNA; (7) the presence of a non-wild-type splicing pattern of a messenger RNA transcript of a programmed cell death-related gene; (8) a non-wild-type level of a programmed cell death protein-like-protein; (9) an allelic loss of a programmed cell death-related gene; and (10) an inappropriate post-translational modification of a programmed cell death protein-like-protein. As described herein, there are a large number of assay techniques known in the art that can be used for detecting lesions in a programmed cell death-related gene. Any cell type or tissue, preferably cerebellar granule neurons, in which programmed cell death-related polypeptides are expressed may be utilized in the prognostic assays described herein.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in programmed cell death protein-like gene (see, e.g., Abravaya et al. (1995) Nucleic Acids Res. 23:675-682). It is anticipated that PCR and/or LCR may be desirable to use as a preliminary amplification step in conjunction with any of the techniques used for detecting mutations described herein.

Alternative amplification methods include self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a programmed cell death-related gene from a sample cell can be identified by alterations in restriction enzyme cleavage patterns of isolated test sample and control DNA digested with one or more restriction endonucleases. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations in a programmed cell death-related molecule can be identified by hybridizing a sample and control nucleic acids, e.g., DNA or RNA, to high density arrays containing hundreds or thousands of oligonucleotides probes (Cronin et al. (1996) Human Mutation 7:244-255; Kozal et al. (1996) Nature Medicine 2:753-759). In yet another embodiment, any of a variety of sequencing reactions known in the art can be used to directly sequence the programmed cell death-related gene and detect mutations by comparing the sequence of the sample programmed cell death-related gene with the corresponding wild-type (control) sequence. Examples of sequencing reactions include those based on techniques developed by Maxim and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Bio/Techniques 19:448), including sequencing by mass spectrometry (see, e.g., PCT Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the programmed cell death-related gene include methods in which protection from cleavage agents is used to detect mismatched bases in RNA/RNA or RNA/DNA heteroduplexes (Myers et al. (1985) Science 230:1242). See, also Cotton et al. (1988) Proc. Natl. Acad. Sci. USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more “DNA mismatch repair” enzymes that recognize mismatched base pairs in double-stranded DNA in defined systems for detecting and mapping point mutations in programmed cell death-related cDNAs obtained from samples of cells. See, e.g., Hsu et al. (1994) Carcinogenesis 15:1657-1662. According to an exemplary embodiment, a probe based on a programmed cell death-related sequence, e.g., a wild-type programmed cell death-related sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in programmed cell death-related genes. For example, single-strand conformation polymorphism (SSCP) may be used to detect differences in electrophoretic mobility between mutant and wild-type nucleic acids (Orita et al. (1989) Proc. Natl. Acad. Sci. USA 86:2766; see also Cotton (1993) Mutat. Res. 285:125-144; Hayashi (1992) Genet. Anal. Tech. Appl. 9:73-79). The sensitivity of the assay may be enhanced by using RNA (rather than DNA), in which the secondary structure is more sensitive to a change in sequence. In a preferred embodiment, the subject method utilizes heteroduplex analysis to separate double-stranded heteroduplex molecules on the basis of changes in electrophoretic mobility (Keen et al. (1991) Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found (Saiki et al. (1986) Nature 324:163); Saiki et al. (1989) Proc. Natl. Acad. Sci. USA 86:6230). Such allele-specific oligonucleotides are hybridized to PCR-amplified target DNA or a number of different mutations when the oligonucleotides are attached to the hybridizing membrane and hybridized with labeled target DNA.

Alternatively, allele-specific amplification technology, which depends on selective PCR amplification, may be used in conjunction with the instant invention. Oligonucleotides used as primers for specific amplification may carry the mutation of interest in the center of the molecule so that amplification depends on differential hybridization (Gibbs et al. (1989) Nucleic Acids Res. 17:2437-2448) or at the extreme 3N end of one primer where, under appropriate conditions, mismatch can prevent or reduce polymerase extension (Prossner (1993) Tibtech 11:238). In addition, it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3N end of the 5N sequence making it possible to detect the presence of a known mutation at a specific site by looking for the presence or absence of amplification.

The methods described herein may be performed, for example, by utilizing prepackaged diagnostic kits comprising at least one probe nucleic acid or antibody reagent described herein, which may be conveniently used, e.g., in clinical settings to diagnosed patients exhibiting symptoms or family history of a disease or illness involving a programmed cell death-related gene.

4. Pharmacogenomics

Agents, or modulators that have a stimulatory or inhibitory effect on programmed cell death-related activity (e.g., programmed cell death-related gene expression) as identified by a screening assay described herein, can be administered to individuals to treat (prophylactically or therapeutically) disorders associated with aberrant programmed cell death-related activity as well as to modulate the phenotype of programmed cell death. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of programmed cell death-like protein, expression of programmed cell death-related nucleic acid, or mutation content of programmed cell death-related genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase deficiency (G6PD) is a common inherited enzymopathy in which the main clinical complication is haemolysis after ingestion of oxidant drugs (antimalarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, an “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drug's target is known (e.g., a programmed cell death-related polypeptide of the present invention), all common variants of that gene can be fairly easily identified in the population and it can be determined if having one version of the gene versus another is associated with a particular drug response.

Alternatively, a method termed the “gene expression profiling”, can be utilized to identify genes that predict drug response. For example, the gene expression of an animal dosed with a drug (e.g., a programmed cell death-related molecule or programmed cell death-related modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a programmed cell death protein like molecule or programmed cell death-related modulator of the invention, such as a modulator identified by one of the exemplary screening assays described herein.

The present invention further provides methods for identifying new agents, or combinations, that are based on identifying agents that modulate the activity of one or more of the gene products encoded by one or more of the programmed cell death-related genes of the present invention, wherein these products may be associated with resistance of the cells to a therapeutic agent. Specifically, the activity of the proteins encoded by the programmed cell death-related genes of the present invention can be used as a basis for identifying agents for overcoming agent resistance. By blocking the activity of one or more of the resistance proteins, target cells, e.g., neurons, will become sensitive to treatment with an agent that, the unmodified target cells were resistant to.

Monitoring the influence of agents or compounds (e.g., drugs) on the expression or activity of a programmed cell death-related polypeptide can be applied in clinical trials. For example, the effectiveness of an agent determined by a screening assay as described herein to increase programmed cell death-related gene expression, protein levels, or upregulate programmed cell death-related activity, can be monitored in clinical trials of subjects exhibiting decreased programmed cell death-related gene expression, protein levels, or downregulated programmed cell death-related activity. Alternatively, the effectiveness of an agent determined by a screening assay to decrease programmed cell death-related gene expression, protein levels, or downregulate programmed cell death-related activity, can be monitored in clinical trials of subjects exhibiting increased programmed cell death-related gene expression, protein levels, or upregulated programmed cell death-related activity. In such clinical trials, the expression or activity of a programmed cell death-related gene, and preferably, other genes that have been implicated in, for example, a programmed cell death-related associated disorder can be used as a “read out” or markers of the phenotype of a particular cell.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of programmed cell death-related polypeptide, expression of programmed cell death-related nucleic acid, or mutation content of programmed cell death-related genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a programmed cell death-related modulator, such as a modulator identified by one of the exemplary screening assays described herein.

5. Monitoring of Effects During Clinical Trials

Monitoring the influence of agents (e.g., drugs, compounds) on the expression or activity of programmed cell death-related genes (e.g., the ability to modulate programmed cell death) can be applied not only in basic drug screening but also in clinical trials. For example., the effectiveness of an agent, as determined by a screening assay as described herein, to increase or decrease programmed cell death-related gene expression, protein levels, or protein activity, can be monitored in clinical trials of subjects exhibiting decreased or increased programmed cell death-related gene expression, protein levels, or protein activity. In such clinical trials, programmed cell death-related expression or activity and preferably that of other genes that have been implicated in for example, a programmed cell death disorder, can be used as a marker of programmed cell death.

For example, and not by way of limitation, genes that are modulated in cells by treatment with an agent (e.g., compound, drug, or small molecule) that modulates programmed cell death-related activity (e.g., as identified in a screening assay described herein) can be identified. Thus, to study the effect of agents on programmed cell death disorders, for example, in a clinical trial, cells can be isolated and RNA prepared and analyzed for the levels of expression of programmed cell death-related genes and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of programmed cell death-related genes or other genes. In this way, the gene expression pattern can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (1) obtaining a preadministration sample from a subject prior to administration of the agent; (2) detecting the level of expression of a programmed cell death-like protein, mRNA, or genomic DNA in the preadministration sample; (3) obtaining one or more postadministration samples from the subject; (4) detecting the level of expression or activity of the programmed cell death-related polypeptide, mRNA, or genomic DNA in the postadministration samples; (5) comparing the level of expression or activity of the programmed cell death-related polypeptide, mRNA, or genomic DNA in the preadministration sample with the programmed cell death-related polypeptide, mRNA, or genomic DNA in the postadministration sample or samples; and (vi) altering the administration of the agent to the subject accordingly to bring about the desired effect, i.e., for example, an increase or a decrease in the expression or activity of a programmed cell death-related polypeptide.

C. Methods of Treatment

The present invention provides for both prophylactic and therapeutic methods of treating a subject at risk of (or susceptible to) a disorder or having a disorder associated with aberrant programmed cell death-related expression or activity. Additionally, the compositions of the invention find use in the treatment of disorders described herein. Thus, therapies for disorders associated with CCC are encompassed herein.

1. Prophylactic Methods

In one aspect, the invention provides a method for preventing in a subject a disease or condition associated with an aberrant programmed cell death-related expression or activity by administering to the subject an agent that modulates programmed cell death-related expression or at least one programmed cell death-related gene activity. Subjects at risk for a disease that is caused, or contributed to, by aberrant programmed cell death-related expression or activity can be identified by, for example, any or a combination of diagnostic or prognostic assays as described herein. Administration of a prophylactic agent can occur prior to the manifestation of symptoms characteristic of the programmed cell death-related aberrancy, such that a disease or disorder is prevented or, alternatively, delayed in its progression. Depending on the type of programmed cell death-related aberrancy, for example, a programmed cell death-related agonist or programmed cell death-related antagonist agent can be used for treating the subject. The appropriate agent can be determined based on screening assays described herein.

2. Therapeutic Methods

Another aspect of the invention pertains to methods of modulating programmed cell death-related expression or activity for therapeutic purposes. The modulatory method of the invention involves contacting a cell with an agent that modulates one or more of the activities of programmed cell death-related polypeptide activity associated with the cell. An agent or compound that modulates programmed cell death-related polypeptide activity can be an agent or compound as described herein, such as a nucleic acid or a protein, a naturally-occurring cognate ligand of a programmed cell death-related polypeptide, a peptide, a programmed cell death-related peptidomimetic, or other small molecule. In one embodiment, the agent stimulates one or more of the biological activities of programmed cell death-related polypeptide. Examples of such stimulatory agents include active programmed cell death-related polypeptide and a nucleic acid molecule encoding a programmed cell death-related polypeptide that has been introduced into the cell. In another embodiment, the agent inhibits one or more of the biological activities of programmed cell death-related polypeptide. Examples of such inhibitory agents include antisense programmed cell death-related nucleic acid molecules and anti-programmed cell death-related polypeptide antibodies.

These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a programmed cell death-related polypeptide or nucleic acid molecule. In one embodiment, the method involves administering an agent (e.g., an agent identified by a screening assay described herein), or a combination of agents, that modulates (e.g., upregulates or downregulates) programmed cell death-related expression or activity. In another embodiment, the method involves administering a programmed cell death-related polypeptide or nucleic acid molecule as therapy to compensate for reduced or aberrant programmed cell death-related expression or activity.

Stimulation of programmed cell death-related activity is desirable in situations in which a programmed cell death-related polypeptide is abnormally downregulated and/or in which increased programmed cell death-related activity is likely to have a beneficial effect. Conversely, inhibition of programmed cell death-related activity is desirable in situations in which programmed cell death-related activity is abnormally upregulated and/or in which decreased programmed cell death-related activity is likely to have a beneficial effect.

This invention is further illustrated by the following examples, which should not be construed as limiting.

### EXPERIMENTAL

### Example 1

Isolation of NARC10 AND NARC16

In neurons, programmed cell death is an essential component of neuronal development (Jacobson et al. (1997) Cell 88:347-354; Pettman et al. (1998) Neuron 20:633-647) and has been associated with many forms of neurodegeneration (Hetts et al. JAMA 279:300-307). In the cerebellum, granule cell development occurs postnatally. The final number of neurons represents the combined effects of additive processes such as cell division and subtractive processes such as target-related programmed cell death. Depolarization due to high concentrations (25 mM) of extracellular potassium (K⁺) promotes the survival of cerebellar granule neurons (CGNs) in vitro. CGNs maintained in serum containing medium with high K⁺ will undergo programmed cell death when switched to serum-free medium with low K⁺ (5 mM) (D'Mello et al. (1993) Proc. Natl. Acad. Sci. USA 90:10989-10993; Miller et al. (1996) J. Neurosci. 16:7487-7495). The resulting programmed cell death has a transcriptional component that can be blocked by inhibitors of new RNA synthesis (Galli et al. (1995) J. Neurosci. 15:1172-1179; Schulz, et al. (1996) J. Neurosci. 16:4696-4706).

As previously disclosed in U.S. patent application Ser. No. 09/692,785, herein incorporated by reference, a Smart Chip™ microarray chip with brain-biased and programmed cell death-enriched clones was constructed by arraying approximately 7300 consolidated EST's from two cDNA libraries cloned from rat frontal cortex and differentiated PC12 cells deprived of nerve growth factor (NGF), and 289 genes that are known markers for the central nervous system and/or programmed cell death. The levels of expression of the genes was monitored at 1, 3, 6, 12, and 24 hours after K⁺ withdrawal. Regulated genes were then sorted by time course expression pattern to identify cellular processes mobilized by cerebellar granule neuron programmed cell death at the RNA level. Included in the analysis were expression profiles of many known pro- and anti-apoptotic regulatory proteins, including transcription factors, Bcl-2 family members, caspases, cyclins, heat shock proteins (HSPs), inhibitors of apoptosis (IAPs), growth factors and receptors, other signal transduction molecules, p53, superoxide dismutases (SODs), and other stress response genes. The time courses of expression of regulated genes induced by K⁺ withdrawal in the presence or absence of serum was compared to time courses of expression induced by glutamate toxicity. A restricted set of relevant genes regulated by multiple models of programmed cell death in cerebellar granule neurons was identified, and these genes included the rat NARC10 and the rat NARC16.

NARC10 encodes an approximately 2 kb mRNA transcript having the corresponding cDNA set forth in SEQ ID NO: 2. This transcript has a 549 nucleotide open reading frame (nucleotides 95-643 of SEQ ID NO: 2), which encodes a 182 amino acid protein (SEQ ID NO: 1). An analysis of the full-length NARC10 polypeptide using the PSORT Protein Localization algorithm predicts a nuclear localization. Prosite program analysis was used to predict various sites within the NARC10 protein. A protein kinase C phosphorylation site was predicted at amino acid 76-78. A casein kinase II phosphorylation site was predicted at amino acid 57-60. N-myristoylation sites were predicted at amino acid 30-35 and 129-134. The NARC10 protein possesses a nucleosome assembly protein domain (amino acid 78-182) and DNA gyrase/topoisomerase IV, subunit A domain (amino acid 92-110) as predicted by HMMer, Version 2.1.1. Screening the NARC10 protein against the ProDom 2000.1 database revealed that the segment of the protein from amino acid 71-128 contained a nucleosome assembly protein 1-like domain and the overlapping segment extending from amino acid 68-114 scored as similar the C. elegans hypothetical CAEEL protein which is a putative nucleosome assembly protein. Another overlapping segment, amino acid 55-119, scored as similar to a 1-phosphatidylinositol-4,5bisphosphate phosphodiesterase.

NARC16 encodes an approximately 3.2 kb mRNA transcript having the corresponding cDNA set forth in SEQ ID NO: 4. This transcript has a 2019 nucleotide open reading frame (nucleotides 145-2163 of SEQ ID NO: 4), which encodes a 672 amino acid protein (SEQ ID NO: 3). A second, brain-restricted isoform of NARC16 that is 1 kb larger than the most abundant form can be detected by northern blotting.

An analysis of the full-length NARC16 polypeptide using the PSORT Protein Localization algorithm predicts a cytoplasmic localization. Prosite program analysis was used to predict various sites within the NARC16 protein. N-glycosylation sites were predicted at amino acid 44-47, 328-331, and 472-475. A cAMP and cGMP-dependent protein kinase phosphorylation site was predicted at amino acid 421-424. Protein kinase C phosphorylation sites were predicted at amino acid 140-142, 148-150, 265-267, 281-283, 345-347, 380-382, 440-442, and 494-496. Casein kinase II phosphorylation sites were predicted at amino acid 100-103, 192-195, 201-204, 261-264, 431-434, 447-450, 475-478, 489-492, and 502-505. N-myristoylation sites were predicted at amino acid 24-29, 114-119, 325-330, and 467-472. An amidation site was predicted for amino acid 494-497. The NARC16 protein ]possesses a starch binding domain (amino acid 3-110) as predicted by HMMer, Version 2.1.1. ProDom analysis indicated that NARC16 contains a glycerophosphoryl diester glycerophosphodiesterase domain (amino acid 321-374), and a glycerophosphoryl diester phosphodiesterase protein T05H10.7-like domain (amino acid 22-138, 270-316, and 574-595). Procaryotic glycerophosphoryl diester glycerophosphodiesterase is a dimeric periplasmically-located enzyme that hydrolyzes deacetylated phospholipids to produce glycerol 3-phosphate and an alcohol (Larson et al. (1983) J. Biol. Chem. 258:5426-5432. Recently, a human protein (MIR 16) with significant similarity to bacterial glycerophosphodiester phosphodiesterase was isolated and is postulated to play a rile in lipid metabolism and G protein signaling (Zheng et al. (2000) Proc. Natl. Acad. Sci. USA 97:3999-4004).

### Example 2

Programmed Cell Death Induced in Cerebellar Granule Neurons by NARC10 and NARC16

A Green Fluorescent Protein (GFP)/NARC16 fusion protein-encoding sequence was cloned into an expression vector and transfected into cultured cerebellar granule neurons. About 10% of cells were transfected based on GFP expression. Cells were stained with Hoechst stain (which stains cell nuclei) and the GFP-positive cells were scored for nuclear condensation. For each condition, greater than 200 GFP-positive cells were scored two times to generate the data provided in FIG. 5. A significant (approximately 10 fold) increase in programmed cell death was detected in GFP/NARC16 transfected cells (GFP.N16) in comparison with negative control GFP transfected cells (GFP) on (lays 3 and 4 after transfection. The increase in programmed cell death seen with expression of the GFP/NARC16 fusion protein was almost as great as that seen for a positive control caspase 9/GFP fusion protein (C9.GFP) (approximately 13 fold) and was greater thin that seen for a positive control GFP/caspase recruitment domain 4 fusion protein (GFP.C4) (approximately 5 fold).

In a related set of experiments, cerebellar granule neurons were transfected with the following enhanced green fluorescent protein (EGFP) expression constructs: Caspase 3-EGFP, Caspase 9-EGFP, EGFP-NARC10, and EGFP-NARC16. 48 hours after transfection, the percentage of GFP positive and GFP negative cells undergoing apoptosis was determined by laser-scanning cytometry. The results are given in Table 1. CGN cells transfected caspase 3-EGFP or caspase 9-EGFP showed a 5.3 and 6.1 fold increase in apoptosis, respectively. EGFP-NARC10 cells showed a 2 fold increase in apoptosis, while EGFP-NARC16 cells showed a 3.5 fold increase in apoptosis.

TABLE 1

| | | Number | Mean % | % of GFP− Cells | % of GFP+ Cells |
|---|---|---|---|---|---|
| Expression | Number of | of GFP + | Transfection | Undergoing | Undergoing |
| Construct | Experiments | Cells | Efficiency | Apoptosis | Apoptosis |
| ΔEGFP | 3 | 19 | 0.04% | 10.07% | — |
| Caspase 3-EGFP | 3 | 936 | 0.78% | 7.88% | 42.15% |
| Caspase 9-EGFP | 2 | 378 | 0.68% | 10.08% | 61.18% |
| EGFP-NARC10A | 3 | 654 | 0.63% | 9.01% | 18.11% |
| EGFP-NARC16 | 3 | 464 | 0.38% | 11.43% | 40.10% |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. An isolated nucleic acid molecule comprising a nucleotide sequence selected from the group consisting of:
a) a nucleotide sequence having at least 70% sequence identity with the nucleotide sequence set forth in SEQ ID NO: 2;
b) a nucleotide sequence having at least 70% sequence identity with the nucleotide sequence set forth in SEQ ID NO: 4;
c) a nucleotide sequence consisting of at least 20 contiguous nucleotides of the nucleotide sequence set forth in SEQ ID NO: 2;
d) a nucleotide sequence consisting of at least 20 contiguous nucleotides of the nucleotide sequence set forth in SEQ ID NO: 4;
e) a nucleotide sequence encoding the amino acid sequence set forth in SEQ ID NO: 1;
f) a nucleotide sequence encoding the amino acid sequence set forth in SEQ ID NO: 3;
g) a nucleotide sequence encoding a fragment of the amino acid set forth in SEQ ID NO: 1, wherein said fragment consists of at least 15 contiguous amino acids of SEQ ID NO: 1;
h) a nucleotide sequence encoding a fragment of the amino acid set forth in SEQ ID NO: 3, wherein said fragment consists of at least 15 contiguous amino acids of SEQ ID NO: 3;
i) a nucleotide sequence encoding a variant of the amino acid sequence set forth in SEQ ID NO: 1, wherein said nucleotide sequence hybridizes to the nucleotide sequence set forth in SEQ ID NO: 2 under stringent conditions;
j) a nucleotide sequence encoding a variant of the amino acid sequence set forth in SEQ ID NO: 3, wherein said nucleotide sequence hybridizes to the nucleotide sequence set forth in SEQ ID NO: 4 under stringent conditions; and
k) a nucleotide sequence complementary to at least one of the nucleotide sequences set forth in a), b), c), d), e), f), g), h), i), or j).

2. The isolated nucleic acid molecule of claim 1, wherein said nucleic acid molecule comprises a nucleotide sequence selected from the group consisting of:
a) the nucleotide sequence set forth in SEQ ID NO: 2;
b) the nucleotide sequence set forth in SEQ ID NO: 4;
c) a nucleotide sequence encoding the amino acid sequence set forth in SEQ ID NO: 1;
d) a nucleotide sequence encoding the amino acid sequence set forth in SEQ ID NO: 3;
e) a nucleotide sequence complementary to a nucleotide sequence of a), b), c), or d).

3. The nucleic acid molecule of claim 1, wherein said nucleic acid molecule further comprises vector nucleic acid sequences.

4. The nucleic acid molecule of claim 1, wherein said nucleic acid molecule further comprises nucleic acid sequences encoding a heterologous polypeptide.

5. A host cell containing the nucleic acid molecule of claim 1.

6. The host cell of claim 5, wherein said host cell is a mammalian host cell.

7. A nonhuman mammalian host cell containing the nucleic acid molecule of claim 1.

8. An isolated polypeptide comprising an amino acid sequence selected from the group consisting of:
a) the amino acid sequence of a fragment of the amino acid sequence set forth in SEQ ID NO: 1, wherein the fragment comprises at least 15 contiguous amino acids of the amino acid sequence set forth in SEQ ID NO: 1;
b) the amino acid sequence of a fragment of the amino acid sequence set forth in SEQ ID NO: 3, wherein the fragment comprises at least 15 contiguous amino acids of the amino acid sequence set forth in SEQ ID NO: 3;
c) the amino acid sequence of a variant of the amino acid sequence set forth in SEQ ID NO: 1, wherein said variant is encoded by a nucleic acid molecule that hybridizes to the complement of the nucleotide sequence set forth in SEQ ID NO: 2 under stringent conditions;
d) the amino acid sequence of a variant of the amino acid sequence set forth in SEQ ID NO: 3, wherein said variant is encoded by a nucleic acid molecule that hybridizes to the complement of the nucleotide sequence set forth in SEQ ID NO: 4 under stringent conditions;
e) the amino acid sequence of a variant of the amino acid sequence set forth in SEQ ID NO: 1, wherein said variant is encoded by a nucleotide sequence having at least 70% sequence identity with the nucleotide sequence set forth in SEQ ID NO: 2; and
f) the amino acid sequence of a variant of the amino acid sequence set forth in SEQ ID NO: 3, wherein said variant is encoded by a nucleotide sequence having at least 70% sequence identity with the nucleotide sequence set forth in SEQ ID NO: 4.

9. The isolated polypeptide of claim 8, wherein said polypeptide comprises an amino acid sequence selected from the group consisting of:
a) the amino acid sequence set forth in SEQ ID NO: 1; and
b) the amino acid sequence set forth in SEQ ID NO: 3.

10. The polypeptide of claim 8, wherein said polypeptide further comprises heterologous amino acid sequences.

11. An antibody which selectively binds to a polypeptide of claim 8.

12. A method for producing a polypeptide selected from the group consisting of:
a) the amino acid sequence of a fragment of the amino acid sequence set forth in SEQ ID NO: 1, wherein the fragment comprises at least 15 contiguous amino acids of the amino acid sequence set forth in SEQ ID NO: 1;
b) the amino acid sequence of a fragment of the amino acid sequence set forth in SEQ ID NO: 3, wherein the fragment comprises at least 15 contiguous amino acids of the amino acid sequence set forth in SEQ ID NO: 3;
c) the amino acid sequence of a variant of the amino acid sequence set forth in SEQ ID NO: 1, wherein said variant is encoded by a nucleic acid molecule that hybridizes to the complement of the nucleotide sequence set forth in SEQ ID NO: 2 under stringent conditions;
d) the amino acid sequence of a variant of the amino acid sequence set forth in SEQ ID NO: 3, wherein said variant is encoded by a nucleic acid molecule that hybridizes to the complement of the nucleotide sequence set forth in SEQ ID NO: 4 under stringent conditions;
e) the amino acid sequence of a variant of the amino acid sequence set forth in SEQ ID NO: 1, wherein said variant is encoded by a nucleotide sequence having at least 70% sequence identity with the nucleotide sequence set forth in SEQ ID NO: 2; and
f) the amino acid sequence of a variant of the amino acid sequence set forth in SEQ ID NO: 3, wherein said variant is encoded by a nucleotide sequence having at least 70% sequence identity with the nucleotide sequence set forth in SEQ ID NO: 4;
wherein said method comprises culturing a host cell comprising a nucleotide sequence encoding said polypeptide under conditions in which the polypeptide is expressed.

13. The method of claim 12, wherein said polypeptide comprises an amino acid sequence selected from the group consisting of:
a) the amino acid sequence set forth in SEQ ID NO: 1; and
b) the amino acid sequence set forth in SEQ ID NO: 3.

14. A method for detecting the presence of a polypeptide in a sample comprising the steps of contacting the sample with a compound that selectively binds to the polypeptide and determining whether the compound binds to the polypeptide in the sample, wherein said polypeptide is selected from the group consisting of:
a) the amino acid sequence of a fragment of the amino acid sequence set forth in SEQ ID NO: 1, wherein the fragment comprises at least 15 contiguous amino acids of the amino acid sequence set forth in SEQ ID NO: 1;
b) the amino acid sequence of a fragment of the amino acid sequence set forth in SEQ ID NO: 3, wherein the fragment comprises at least 15 contiguous amino acids of the amino acid sequence set forth in SEQ ID NO: 3;
c) the amino acid sequence of a variant of the amino acid sequence set forth in SEQ ID NO: 1, wherein said variant is encoded by a nucleic acid molecule that hybridizes to the complement of the nucleotide sequence set forth in SEQ ID NO: 2 under stringent conditions;
d) the amino acid sequence of a variant of the amino acid sequence set forth in SEQ ID NO: 3, wherein said variant is encoded by a nucleic acid molecule that hybridizes to the complement of the nucleotide sequence set forth in SEQ ID NO: 4 under stringent conditions;
e) the amino acid sequence of a variant of the amino acid sequence set forth in SEQ ID NO: 1, wherein said variant is encoded by a nucleotide sequence having at least 70% sequence identity with the nucleotide sequence set forth in SEQ ID NO: 2; and
f) the amino acid sequence of a variant of the amino acid sequence set forth in SEQ ID NO: 3, wherein said variant is encoded by a nucleotide sequence having at least 70% sequence identity with the nucleotide sequence set forth in SEQ ID NO: 4.

15. The method of claim 14, wherein the compound which binds to the polypeptide is an antibody.

16. A kit comprising a compound which selectively binds to a polypeptide and instructions for use, wherein said polypeptide is selected from the group consisting of:
a) the amino acid sequence of a fragment of the amino acid sequence set forth in SEQ ID NO: 1, wherein the fragment comprises at least 15 contiguous amino acids of the amino acid sequence set forth in SEQ ID NO: 1;
b) the amino acid sequence of a fragment of the amino acid sequence set forth in SEQ ID NO: 3, wherein the fragment comprises at least 15 contiguous amino acids of the amino acid sequence set forth in SEQ ID NO: 3;
c) the amino acid sequence of a variant of the amino acid sequence set forth in SEQ ID NO: 1, wherein said variant is encoded by a nucleic acid molecule that hybridizes to the complement of the nucleotide sequence set forth in SEQ ID NO: 2 under stringent conditions;
d) the amino acid sequence of a variant of the amino acid sequence set forth in SEQ ID NO: 3, wherein said variant is encoded by a nucleic acid molecule that hybridizes to the complement of the nucleotide sequence set forth in SEQ ID NO: 4 under stringent conditions;
e) the amino acid sequence of a variant of the amino acid sequence set forth in SEQ ID NO: 1, wherein said variant is encoded by a nucleotide sequence having at least 70% sequence identity with the nucleotide sequence set forth in SEQ ID NO: 2; and
f) the amino acid sequence of a variant of the amino acid sequence set forth in SEQ ID NO: 3, wherein said variant is encoded by a nucleotide sequence having at least 70% sequence identity with the nucleotide sequence set forth in SEQ ID NO: 4

17. A method for detecting the presence of a nucleic acid molecule in a sample, the method comprising the steps of contacting the sample with a nucleic acid probe that hybridizes to the nucleic acid molecule under stringent conditions, and determining whether the nucleic acid probe binds to a nucleic acid molecule in the sample, wherein said nucleic acid molecule is selected from the group consisting of:
a) a nucleotide sequence having at least 70% sequence identity with the nucleotide sequence set forth in SEQ ID NO: 2;
b) a nucleotide sequence having at least 70% sequence identity with the nucleotide sequence set forth in SEQ ID NO: 4;
c) a nucleotide sequence consisting of at least 20 contiguous nucleotides of the nucleotide sequence set forth in SEQ ID NO: 2;
d) a nucleotide sequence consisting of at least 20 contiguous nucleotides of the nucleotide sequence set forth in SEQ ID NO: 4;
e) a nucleotide sequence encoding the amino acid sequence set forth in SEQ ID NO: 1;
f) a nucleotide sequence encoding the amino acid sequence set forth in SEQ ID NO: 3;
g) a nucleotide sequence encoding a fragment of the amino acid set forth in SEQ ID NO: 1, wherein said fragment consists of at least 15 contiguous amino acids of SEQ ID NO: 1;
h) a nucleotide sequence encoding a fragment of the amino acid set forth in SEQ ID NO: 3, wherein said fragment consists of at least 15 contiguous amino acids of SEQ ID NO: 3;
i) a nucleotide sequence encoding a variant of the amino acid sequence set forth in SEQ ID NO: 1, wherein said nucleotide sequence hybridizes to the nucleotide sequence set forth in SEQ ID NO: 2 under stringent conditions;
j) a nucleotide sequence encoding a variant of the amino acid sequence set forth in SEQ ID NO: 3, wherein said nucleotide sequence hybridizes to the nucleotide sequence set forth in SEQ ID NO: 4 under stringent conditions; and
k) a nucleotide sequence complementary to at least one of the nucleotide sequences set forth in a), b), c), d), e), f), g), h), i), or j).

18. The method of claim 17, wherein the sample comprises mRNA molecules.

19. A kit for use in the method of claim 17, wherein said kit comprises a compound which selectively hybridizes to at least one nucleic acid molecule of claim 1 and instructions for use.

20. A method for identifying a compound which binds to a polypeptide of claim 8, said method comprising the steps of:
a) contacting a polypeptide, or a cell expressing a polypeptide of claim 8 with a test compound; and
b) determining whether the polypeptide binds to the test compound.

21. The method of claim 20, wherein the binding of the test compound to the polypeptide is detected by a method selected from the group consisting of:
a) detection of binding by direct detecting of test compound/polypeptide binding;
b) detection of binding using a competition binding assay;
c) detection of binding using an assay for programmed cell death protein-like-mediated activity.

22. A method for modulating the activity of a polypeptide of claim 8 comprising contacting a polypeptide or a cell expressing a polypeptide of claim 8 with a compound which binds to the polypeptide in a sufficient concentration to modulate the activity of the polypeptide.

23. A method for identifying a compound which modulates the activity of a polypeptide of claim 8, comprising:
a) contacting a polypeptide of claim 8 with a test compound; and
b) determining the effect of the test compound on the activity of the polypeptide to thereby identify a compound which modulates the activity of the polypeptide.
